(19) United States
(12) Patent Application Publication
(10) Pub. No.: US 2019/0134263 A1
(43) Pub. Date: May 9, 2019

(54) System and Method for Vascularized Biomimetic 3-D tissue Models

(71) Applicants: Cheul H Cho, Whippany, NJ (US); Ali Hussain, Newark, NJ (US); George Collins, Maplewood, NJ (US); Divya Ranendran, Stamford, CT (US)

(72) Inventors: Cheul H Cho, Whippany, NJ (US); Ali Hussain, Newark, NJ (US); George Collins, Maplewood, NJ (US); Divya Ranendran, Stamford, CT (US)

(21) Appl. No.: 14/003,062

(22) PCT Filed: Mar. 1, 2012
(86) PCT No.: PCT/US12/27348

§ 371 Date: Oct. 23, 2017

Related U.S. Application Data
(60) Provisional application No. 61/448,483, filed on Mar. 2, 2011.
(60) Provisional application No. 61/557,652, filed on Nov. 9, 2011.

(51) Int. Cl. A61L 27/26

(52) U.S. Cl. CPC A61L 27/26

(57) ABSTRACT

The present invention relates to a vascularized three dimensional construct for thick tissue, a process for making the construct and to the use of the construct in tissue regeneration and repair and in drug development. The three-dimensional (3-D) tissue technology is used to generate vascularized, biomimetic tissue models in vitro utilizing a biodegradable nanofiber scaffold. The culture system allows the maintenance of long-term survival and function of liver and heart cells. The system utilizes a novel approach to generate structures that mimic in vivo tissue architecture. The system provides a microenvironment for forming 3-D microvascular networks within the nanofiber scaffolds.

### FIELD OF THE INVENTION

The present invention relates to a vascularized three dimensional construct for thick tissue, a process for making the construct and to the use of the construct in tissue regeneration and repair and in drug development.

### BACKGROUND OF THE INVENTION

Tissue engineering has emerged as a novel therapeutic approach for tissue repair/regeneration and in vitro models for drug testing. Three-dimensional (3D) scaffold-based tissue-engineered constructs possess fundamental advantages over traditional 2D culture approaches by allowing cells to organize into structures that mimic their in vivo architecture. Despite significant progress in this field, current tissue models such as liver and heart are not yet able to stably maintain functional characteristics for therapeutic purposes.

A major challenge in the engineering of any tissue is the incapability of providing sufficient blood supply to the damaged tissue immediately post implantation. Engineering grafts for thick complex tissues such as cardiac and hepatic tissues require adequate vasculature to sustain physiological requirements since the diffusion limit for oxygen is 100-200 μm.

This challenge is intensified when engineering a physiological demanding tissue such as the myocardium. The native myocardial tissue is supplied by rich vasculature which is necessary to quench the immense demand for oxygen and nutrients required for its continuous vigorous contractile activity. In addition to serving as conduits for blood supply, endothelial cells are vital for promoting cardiomyocyte survival and function. Neureglin secreted by endothelial cells affect cardiomyocyte survival, proliferation and hypertrophic growth through the phosphatidylinositol-3-kinase-AKt pathway. Furthermore, cardiac endothelial cells influence cardiomyocyte contractility by the secretion of numerous modulators, such as nitric oxide which affects cardiomyocyte inotropism, endothelin causes cardiomyocyte constriction and platelet derived growth factor (PDGF) affect cardiomyocyte development.

Several approaches have been investigated to vascularize cardiac grafts. Capsi et al. seeded human Embryonic Stem Cell derived endothelial cells (hESC-EC) or Human Umbilical Vein EC (HUVEC) on PLLA/PLGA porous scaffolds along with embryonic fibroblasts and were able to depict some vascularization. Radisic et al. covalently immobilized angiogenic factors VEGF and angiopoietin-1 onto collagen sponges resulting in an increase in infiltration of murine embryonic heart endothelium cells into the scaffold and an elongated cellular morphology. Okano et al. were able to show in growth of vasculature from HUVEC sheets into fibroblast sheets that were stack on top of each other. Dvir et al. were successful in showing that maturing their cardiac Matrigel incorporated constructs on a blood rich membrane, rat omentum, enabled the formation of functional blood vessel networks. Matrigel is a biological mixture of the basement membrane extracted from rat chondrosarcoma ECM, which makes it unfavorable for patient use. While this previous research has led to limited success, the goal of engineering a favorable technique to generate 3-D vasculature networks within scaffolds remains elusive.

Engineering a system that will help repopulate the infarct region with functional cardiomyocyte has experienced much progress in the experimental phase. Several approaches to the problem are being investigated varied in the cell species used, scaffold design and biomaterial, and the biological, physical and chemical cues used to culture the cells in vitro. The technology of cell sheeting pioneered by Okano et al. has demonstrated the opportunity of creating a 3-D construct by layering cell sheets cultured on temperature-sensitive polymers. Zimmermann et al. investigations indicate that mixing cardiomyocytes from neonatal rats with collagen I and other ECM factors that are cast in circular molds can improve heart function in vivo. Vacanti et al. utilized electrospun polycaprolactone nanofibers to culture cardiomyocytes and have shown that that cardiomyocytes attached to the meshes and expressed functional cardiomyocyte proteins. These advances still have to overcome several pivotal problems such as cell sourcing, in vitro culture conditions, vascularization of the graft, and designing the hierarchical architecture and intricate 3-dimensional geometry of cardiac tissue.

Drug metabolism is vital for pharmacology for many reasons. Firstly, the blood level is controlled by the metabolism of drugs and therefore influences it therapeutic and/or possible toxic effects. Second, some drugs require biotransformation into active metabolites for therapeutic applications. Third, it may generate highly reactive metabolites which after covalent binding to either proteins or nucleic acids, may generate serious side effects and pathologies. Lastly, drugs may modify the response of the organism to other compounds which are biotransformed by these enzyme systems.

Drug metabolism is generally divided in to two phases. Phase I or functionalization reactions and Phase II or conjugative reactions. The biotransformation pathway is usually determined by either Phase I or II or both. Major part of biotransformation occurs through Phase I reactions mainly through oxidation performed by the microsomal mixed-function oxidase system also known as the cytochrome P450 (CYP) family of enzymes and in small percentage by other groups such as flavin-containing monoxygenase. In phase II reactions involves a wide range of enzymes along with an ‘activated’ co-factor or a substrate derivative resulting in a water soluble final product which is excreted through bile or urine.

The main aim of drug metabolism studies is to determine what happens in humans due to the action of the drug and/or its metabolites. These studies are also important to understand the pathway of metabolism of compounds in man, to determine the efficacy, duration of action and toxicity of the drug. Liver is the principal target organ for the obnoxious effects of xenobiotics, in addition to being the main organ responsible for drug metabolism. Metabolism also occurs in other organs such as kidney, lungs, intestine, skin and brain to a lesser extent

In order to evaluate hepatic drug intake and metabolism, microsomal cytochrome P450 induction, drug interactions, hepatotoxicity and cholestasis to improve drug development and discovery process, it is vital to maintain a well-differentiated hepatocyte culture for prolonged periods of time with intact phase I and phase II biotransformation capacities. However, availability of healthy liver samples are limited by ethical constraints and the difficulty of finding homogeneous group of subjects to perform studies. Studies cannot be conducted on humans due to ethical and practical constraints. It is ethically not acceptable to take a liver sample from a healthy volunteer considering the risks of the procedure. Hence, the samples that are generally available are more or less diseased tissues, which make interpretation of data and extrapolation to the normal human difficult. The development of a successful in vitro liver model will minimize the use of laboratory animal and reduces post market withdrawal of drugs.

Electrospinning is a fabrication technique by which submicron to nanometer fibers are produced as a non-woven mat from an electrostatically driven jet of polymer solution. Electrospinning is currently being extensively studied for tissue engineering applications because of its ability to produce nano-structures with a very high surface area to mass ratio (40 to 100 m²/g). In addition, the fibrous structure forms a network of interconnected voids that provides an environment that is similar to the in vivo ECM. A variety of synthetic or natural polymers have been used to fabricate nanofibrous scaffolds using electrospinning technique. Chitosan, a natural polysaccharide, is widely used in tissue engineering because of its biocompatibility, biodegradability, non-toxicity, and its pH dependent solubility facilitating its processing into micro- and nano-scaffolds. The chemical structure of chitosan is similar to the glycosaminoglycans in the extracellular matrix, and its hydrophilicity enhances its interaction with growth factors, cellular receptors, and adhesion proteins. The electrospinning of pure chitosan fibers has been reported. Although electrospun chitosan nanofiber scaffolds have been recently studied in many tissue engineering applications, there have been no reports of chitosan nanofiber scaffolds for cardiac tissue engineering applications.

### SUMMARY OF THE INVENTION

This invention relates to the use of three-dimensional (3-D) tissue technology to generate vascularized, biomimetic tissue models in vitro utilizing a biodegradable nanofiber scaffold. The culture system allows the maintenance of long-term survival and function of liver and heart cells. The system utilizes a novel approach to generate structures that mimic in vivo tissue architecture. The system provides a microenvironment for forming 3-D microvascular networks within the nanofiber scaffolds. Furthermore, in one embodiment, the system utilizes a unique system that does not require tumor-derived Matrigel for vascularization and enable maintenance of capillary-like structure for long-term. In another embodiment, the system utilizes nanofiber technology using natural polysaccharide chitosan polymer to mimic in vivo-like extracellular matrix. The 3-D models that have long-term, stable function can be used as reliable tissue models for drug screening and tissue regeneration.

### BRIEF DESCRIPTION OF THE DRAWINGS

So that those having ordinary skill in the art will have a better understanding of how to make and use the disclosed systems and methods, reference is made to the accompanying figures wherein:

FIG. 1 are SEM images of vacuum dried electrospun chitosan prepared from 8% chitosan solution dissolved in trifluoroacetic acid/methylene chloride (80:20) solution (A) 5,000× original magnification, (B) 50,000× original magnification, and (C) fiber diameter distribution of the chitosan nanofibers. (D) Photography of the chitosan nanofiber mat;

FIG. 2 illustrates fibronectin (FN) adsorption on chitosan (A) Relative fluorescence intensity of fibronectin adsorbed on chitosan coated tissue culture dishes at various fibronectin concentrations by immunofluorescence staining, (B and C) phase and immunofluorescence staining for anti-fibronectin of chitosan nanofibers adsorbed by fibronectin solution (10 μg/ml) at 200× original magnification;

FIG. 3 illustrates morphology, Vinculin (focal adhesion) expression, and F-actin distribution of endothelial cells (A and B) and cardiomyocytes (C and D) cultured on chitosan or chitosan-FN coated tissue culture dishes (Day 2). Phase-contrast and immunofluorescence staining images for vinculin (green), F-actin (red), and DAPI (blue) of endothelial cells and cardiomyocytes cultured on chitosan or fibronectin coated chitosan membrane surfaces. Neonatal cardiomyocytes (CM), microvascular endothelial cells (EC). 200× original magnification;

FIG. 4 shows (A-C) Live/dead cell staining of 3T3-J2 fibroblasts seeded on chitosan-FN nanofiber scaffolds after 4 days of culture, calcein staining for live cells and ethidium homodimer for dead cells, 200× original magnification, (D-F) SEM images of fibroblasts, cardiomyocytes, and endothelial cells cultured on Chitosan-FN nanofiber scaffolds after three weeks of culture, fibronectin (FN);

FIG. 5 displays morphology and phenotypic characteristics of cardiomyocytes on 2-D Chitosan-FN film. (A, D) Cardiomyocytes cultured alone, (B, E) Cardiomyocytes co-cultured with 3T3-J2 fibroblasts, and (C, F) Cardiomyocytes co-cultured with microvascular endothelial cells on day 7. Cardiomyocytes were immunostained for α-sarcomeric actin (SA-actin) and connexin-43 (Cx43) gap junction expression, neonatal cardiomyocytes (CM), 3T3-J2 fibroblasts (FB), microvascular endothelial cells (EC). 200× original magnification;

FIG. 6 illustrates morphology and phenotypic characteristics of cardiomyocytes in 3-D Chitosan-FN nanofiber scaffolds, (A, D) Cardiomyocytes cultured alone, (B, E) Cardiomyocytes co-cultured with 3T3-J2 fibroblasts, and (C, F) Cardiomyocytes co-cultured with microvascular endothelial cells on day 19, Cardiomyocytes were immunostained for α-sarcomeric actin (SA-actin) and connexin-43 (Cx43) gap junction expression, neonatal cardiomyocytes (CM), 3T3-J2 fibroblasts (FB), microvascular endothelial cells (EC). 200× original magnification;

FIG. 7 displays (A) The spinnability of the chitosan solution and its relationship between the viscosity and solution stirring time, the most spinnable time point is 12-15 hours post dissolution, (B) scanning electron mcirograph of electrospun 8% chitosan from trifluoroacetic acid and methyelne chloride (80:20 v/v) at magnification 25,000×;

FIG. 8 The stress-strain profile of electrospun chitosan under uni-axial tensile stress;

FIG. 9 depicts (A) scanning electron micrograph of electrospun chitosan fibers after 28 days of PBS incubation, showing the increased fiber diameter distribution at magnification 20,000×, (B) is a graphic representation of the widening fiber diameter distribution during 0, 1, 7, 14, 21, 28 days of PBS incubation, (C) average fiber diameter and standard deviation during PBS incubation;

FIG. 10 visually depicts (A) dry weight loss of electrospun chitosan nanofibers during the in vitro degradation assay in PBS solution at 37° C. with 4 mg/ml lysozyme; (B) scanning electron micrograph of the electrospun chitosan nanofibers after 28 days incubation in 4 mg/ml lysozyme solution at magnification 20,000×;

FIG. 11 displays FTIR spectra of (1) chitosan powder (2) film and (3) electrospun fibers;

FIG. 12 graphically depicts (A) DSC results for chitosan powder and electrospun nanofibers (1) first heating cycle (2) second heating cycle, (B) TGA weight loss profile for chitosan powder and electrospun nanofibers;

FIG. 13 displays X-ray diffractograms of chitosan (1) powder, (2) film and (3) electrospun;

FIG. 14 illustrates endothelial cell tube formation assay using Matrigel in 2-D culture with 2-D capillary-like tube formation of endothelial cells (LSEC) on Matrigel on day 1. (A) phase, (B) calcein, (C) DAPI, and (D) SEM image, original magnification: 200× for A, B, C; 1,000× for D;

FIG. 15 displays comparison of the tube formation of endothelial cells (LSEC) on fibronectin coated 3-D chitosan nanofibers without Matrigel (A) and with Matrigel (B) on days 14 and 21, cells were stained with green fluorescent calcein. Images at 40× original magnification;

FIG. 16 shows effect of seeding density of endothelial cells (LSEC) on vascularization within 3-D nanofiber scaffolds without Matrigel on days 1, 7, 14, and 21, cells were stained with green fluorescent calcein AM;

FIG. 17 shows tube formation of endothelial cells (LSEC) in 3-D chitosan nanofibers without Matrigel on day 14. (A and C) Calcein staining at high (200×) and low (40×) magnification; (B and D) SEM images. Original magnification: 200× for A, 40× for B, 2,500× for C, and 10,000× for D;

FIG. 18 displays formation of microvascular networks of endothelial cells cultured within 3-D nanofiber scaffolds without Matrigel for 14 days, LSEC-seeded scaffolds were fixed and cut into piece for further analysis, the cells were stained with Safranin-O dye for cells and ECM staining;

FIG. 19 shows human liver cells (HepG2) cultured alone (A, C) and cocultured with LSEC for vascularization (B, D), Day 9, Safranin-O dye staining (A and B; 200× magnification) and SEM images (C and D; 2500× magnification);

FIG. 20 displays pseudo-color intensity images of transient calcium ion flow of cardiomyocytes in tri-culture system (cardiomyocytes+fibroblasts+endothelial cells) after 7 days of culture on 3-dimensional chitosan nanofibers, the images are pseudocolored according to fluorescence intensity, with red representing high Ca2+ concentrations and blue representing low Ca2+ concentrations, cell types used are rat neonatal cardiomyocytes, mouse 3T3-J2 fibroblast and rat liver sinusoidal endothelial cells, 200× original magnification, the green-fluorescent calcium indicator, fluo-4 AM (Invitrogen), was used to monitor calcium ion flow;

FIG. 21 shows morphological characteristics of hepatocytes in monoculture and co-culture;

FIG. 22 shows morphological characteristics 3-D-SEM images of co-cultured 3-D liver model for Day 14;

FIGS. 23a and 23b show urea synthesis in 2D and 3D culture systems respectively;

FIGS. 24a and b show albumin secretion in short term and long term 2-D and 3-D cultures respectively; and

FIG. 25 shows the comparison of CYP450 activity for short term and long term culture.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

The present invention utilizes three-dimensional (3-D) tissue technology to generate vascularized, biomimetic liver and heart models in vitro utilizing a nanofiber scaffold. The culture system of multiple embodiments of the present invention allows the maintenance of long-term survival and function of cells such as liver and heart cells. Said embodiments utilize a novel approach to generate structures that mimic in vivo tissue architecture. Said embodiments provide a microenvironment for forming 3-D microvascular networks within the nanofiber scaffolds. Furthermore, embodiments of the present invention utilize a unique system that does not require tumor-derived Matrigel for vascularization and enable maintenance of capillary-like structure for long-term Significance. The system utilizes nanofiber technology and can utilize, for example, natural polysaccharide chitosan polymer to mimic in vivo-like extracellular matrix in certain aspects of the present invention. The FDA approved chitosan is widely used in the field of biomedical science and have been used clinically. The 3-D models that have long-term, stable function can be used as reliable tissue models for drug screening and tissue regeneration.

More particularly, this invention relates to a nanofiber 3-dimensional (3-D) scaffold which comprises an electrospun polymer fiber. The scaffold can be a 3-dimensional (3-D) scaffold wherein the electrospun polymer fiber coated with surface coating molecule. The fiber can be chosen from those known in the art, for example, as collagen, gelatin, chitosan and synthetic polymers such as PLLA, PLGA, PCL (polycaprolactone). The surface coating molecule is chosen from one known in the art such as fibronectin, laminin, poly-lysine, or glycosaminglycans, more particularly fibronectin. The glycosaminglycans can be ionically or covalently coated with a cross-linking reagent wherein the cross-linking reagent is one known in the art such as glutaraldehyde, genipin or EDC/sulfoNHS.

Another embodiment of the invention relates to a method of preparing electrospun chitosan. More particularly, it relate to the starting chitosan solution for the preparation of the nanofibers. A favored concentrate is a 4-12% solution of chitosan in solvent. Concentrations of other electrospun polymer fiber can be chosen according to their know properties. A favored solvent for chitosan fibers is trifluoroacetic acid and methylenechloride.

Yet another embodiment of the invention relates to a method to coat the surfaces of chitosan nanofibers with fibronectin. The method comprises the steps of sterilizing the chitosan nanofibers; incubating the nanofibers in a fibronectin solution; and aspirating the excess fibronectin. The concentration of the fibronectin in solution is preferably solution is from 1 to 50 μg/ml of fibronectin in deionized water or a buffer solution.

Another embodiment of the invention relates to a method to coat the surfaces of chitosan nanofibers with glycosaminglycans by ionically or covalently using cross-linking reagents. Such cross-linking agents are preferably glutaraldehyde, genipin, and EDC/Sulfo-NHS.

Another embodiment of the invention relates to a method to maintain the long-term function of cardiomyocytes or hepatocytes which comprises co-culturing the cardiomyocytes or hepatocytes with 3T3-J2 fibroblasts in 2-D nanofibers culture or 3-D nanofiber culture. In addition, the method can be carried out by tri-culturing the cardiomyocytes with fibroblasts and endothelial cells in 2-D nanofiber culture 3-D nanofiber culture. The fibroblasts can be rat, mouse, and human fibroblasts, or selected from 3T3-J2 fibroblasts, NIH-3T3 fibroblasts, or embryonic fibroblasts. Endothelial cells are, for example, liver sinusoidal endothelial cells (LSEC), HUVEC, microvascular endothelial cells, or aortic endothelial cells from vertebrates such as rat, mouse, and human endothelial cells.

Yet another embodiment of the invention relates to a method for forming 2-D or 3-D microvascular networks which comprises seeding a 2-D nanofibers or a 3-D nanofiber scaffold with cardiomyocytes or hepatocytes and incubating the culture.

Yet another embodiment of the invention relates to a drug screening model. An ideal in vitro drug screening model must maintain well-differentiated hepatocyte culture for prolonged periods of time with intact phase I and phase II biotransformation capacities, it must mimic the natural liver functions and architecture, it must be able to evaluate hepatic drug intake and metabolism, microsomal cytochrome P450 induction, drug interactions, hepatotoxicity and cholestasis. This would minimize the use of laboratory animal and reduces post market withdrawal of drugs.

Drug biotransformation is one of the most important factors which is used to identify the overall therapeutic new therapeutic agent. The drug discovery and development process is long and often hindered by unanticipated problems. It involves a series of investigational phases, starting by demonstrating the efficacy in experimental cell and animal models, followed by a concluding demonstration of safety and efficacy in humans. Drugs can fail at any point in this investigating timeline. Therefore, the study of various aspects of metabolism and toxicity of xenobiotics, especially those of new drugs and new chemical entities with the aid of the use of in vitro and in vivo systems, becomes an essential part of drug development and discovery process. There may be various reasons for failure however the most common is often due to unacceptable toxicity in one or more animal species or in clinical trials, which leads to terrible losses in cost and time. This can be avoided if hepatotoxicity is identified earlier in the development process namely, the pre-clinical stage. However, since only a small quantity of drug is available for testing in the early stages, the only possible approach is in vitro testing.

Therefore, it is vital to mimic the structural organization of natural liver for multi-functionality and maintenance of hepatocytes in order to create a human-relevant in vitro drug screening system.

Thus 3-D chitosan nanofiber scaffolds have been fabricated using an electrospinning technique and test for the feasibility of using 3-D chitosan nanofibers as scaffolds for cardiac tissue engineering applications. It has been demonstrated that the chitosan nanofibers retain their cylindrical morphology in long-term cell cultures and exhibit good cellular attachment and spreading in the presence of adhesion molecule, fibronectin.

In both 2-D and 3-D cultures on chitosan constructs, cardiomyocyte-fibroblasts co-cultures resulted in polarized cardiomyocyte morphology with high levels of SA-actin and Cx43 expression over long-term culture periods. In addition, the fibroblasts co-cultures demonstrated synchronized contractions involving large tissue-like cellular networks, indicating the maintenance of long-term and stable function of cardiomyocytes gap junctions. Thus, 3-D chitosan nanofibers can be used as a potential scaffold that can retain cardiomyocyte morphology and function.

Cardiac fibroblasts are the most abundant non-cardiomyocyte cells in the mature heart. Their functions include deposition of the extracellular matrix (ECM), paracrine signaling and propagation of the electrical stimuli. Murine 3T3-J2 fibroblasts cell line for were used for cardiac co-cultures because of their easy access, propagation, and high induction of epithelial cell functions (e.g. hepatocytes). A key feature was the co-culturing of cardiomyocytes with either fibroblasts or endothelial within a 3-D scaffold for long-term functionality of the cardiomyocytes. SA-actin expression was solely found in cardiomyocytes and was expressed the most in the fibroblasts co-cultures. Cx43 is the gap junction protein that is mainly found in ventricular cardiomyocytes The Cx43 mediates fibroblasts heterogeneous coupling, such as between cardiomyocytes and fibroblasts. These gap junctions with fibroblasts are known to propagate electrical stimuli for 100 from both the 2-D and 3-D cultures indicate that fibroblasts co-cultures resulted in high levels of SA-actin and Cx43 expression, suggesting fibroblasts are essential in maintaining cardiomyocytes viability and function in vitro.

Co-culture of primary rat hepatocytes with other cell types can maintain liver-specific functions for several weeks in vitro. Table 1 lists representative cell types used in co-culture with rat hepatocytes for long term hepatic function.

TABLE 1

| Liver derived Cells | Non-liver derived Cells |
|---|---|
| Rat Liver epithelial (presumed | Bovine aortic endothelia |
| biliary origin) | Canin kidney epithelia |
| Stellate | Chinese hamster pithelia |
| Sinusoidal endothelial | |
| Kupffer | Embryonic murine (3T3, C3H, 10T) |
| ‘Non-parenchymal” fraction of | Human fibroblast |
| isolated population | Human lung epithelia |
| | Human venous endothelia |
| | Monkey kidney epithelia |
| | Rat dermal fibroblast |

The co-culture system has also been found to retain total CYP P450 content, triglyceride and urea synthesis, phase I and II biotransformation reactions, normal bile acid transport roperties and the ability to secrete α2-macroglobulin after stimulation by cytokines and enhance gap junctional intercellular communications.

The higher viability and maintenance of function of hepatocytes co-cultured with other celltypes requires intercellular contact or otherwise known as the heterotypic cell-cell interactions. The hepatocyte morphology and functions vary according to the co-culture cell type. In vivo hepatocytes are large, compact polyhedral cells with a round nuclei and prominent nucleoli but when isolated and culture alone, they lose their function and also many of their characteristic features. The cell borders become indistinct and the actin cytoskeleton undergoes rearrangements leading to a ‘fibroblast-like’ appearance, which eventually leads to necrosis and cell death. However, when hepatocytes are grown in co-cultures, it exhibits stereotypical polygonal morphology with distinct nuclei and nucleoli, distinct cell-cell borders and a visible bile canalicular network for many weeks. The differences in morphologies and function with different co-cultures may be due to the different proliferative responses of hepatocytes in the various co-cultures. It may also be due to variations in cell signaling, growth factor release, ECM deposition and protein production. Phase contrast and SEM images of 2-D and 3-D co-culture models repectively, showed formation of hepatocyte colonies surrounded by fibroblast cells. The 3-D models showed increased urea synthesis as compared to 2-D models which were cultured beyond 2 weeks.

Another advantage of the system of the invention is the use of electrospun chitosan to create nano- to micro-sized fibers that reproduce the spatial dimensionality of the fibrous component of the ECM. The fact that cardiomyocytes are able to survive and contract on chitosan nanofibers is apparently not known in the art. It is projected that these mats can be layered on top of each other to create a thick tissue-like structure composed of cardiomyocytes, fibroblasts and endothelial cells. The fibroblasts enhance the electrical synchronization of the cardiomyocytes, while the endothelial cells have the potential to facilitate vascularization into the graft. Chitosan can interact electrostatically with cells since cells carry an overall slightly negative surface charge and chitosan's free amine group can become protonated allowing ionic interactions. The data suggests that cells cultured on chitosan surfaces maintained rounded morphology with poor cell adhesion. However, cells cultured on fibronectin coated chitosan surfaces exhibited typical elongated shape with improved cell adhesion. Fibronectin is a large ECM glycoprotein which facilitates cell adhesion and spreading via α5β1 and αvβ3 integrin receptors in cells. The integrins recognize and interact with RGD cell adhesion domains initiating cell signaling pathways that control cell survival, proliferation, differentiation, and remodeling of the ECM. The amine groups present in chitosan are engaged in fibronectin adsorption. Functional activity of fibronectin is conserved because of minimum protein unfolding conserving the cell adhesion sites.

In one embodiment of the invention it has been demonstrated that chitosan nanofibers can be used as scaffolds for the development of 3-D cardiac tissue constructs that more closely resemble native heart tissue. The cardiac co-culture model of the invention is a promising system for the maintenance of long-term survival and function of cardiomyocytes. The engineered 3-D cardiac co-culture model using chitosan nanofiber scaffolds can be useful for the design and improvement of engineered tissues for the repair of myocardial infarcts, tissue engineering applications, and drug testing.

Human liver cells and human heart cells are similar in the sense of relatively thick, highly vascularized tissue with large oxygen consumption needs. Therefore co-cultures and tri-cultures of involving both cell types as described herein can utilize either type of cell. One embodiment of the present invention that involves a 3-D construct that utilizes the 3D seeding methods described above comprises 3T3-J2 fibroblasts, NIH-3T3 fibroblasts, embryonic fibroblasts, etc. along with endothelial cells from rat, mouse, or humans. Also in one embodiment, the endothelial cells used are liver sinusoidal endothelial cells (LSEC), HUVEC, microvascular endothelial cells, aortic endothelial cells, etc. Another embodiment of the invention is a method to maintain the long-term function of liver hepatocytes by co-culturing with 3T3-J2 fibroblasts in 3-D nanofiber cultures. Further embodiments allow for a method to maintain the long-term function of liver hepatocytes by tri-culturing with 3T3-J2 fibroblasts and endothelial cells in 3-D nanofiber cultures using the protocol described above for experimental cardiomyocyte scaffolds. The method also embraces the use of fibroblasts from rat, mouse, and human sources.

This methodology allows for embodiments of the present invention utilizing hepatocytes or cardiomyocytes to create 3-D microvascular network using nanofiber scaffolds with or without Matrigel as well as 3-D microvascular networks within the nanofiber scaffolds by biophysical and biochemical factors. Also, the methods described herein all for coating the nanofibers with Matrigel or collagen gel amongst other combinations.

Furthermore, as to the nanoscaffolds themselves, embodiments of the present invention allow for a method of producing nanofibers where chitosan solution is 4-12% in solvent with the solvent further comprising trifluoroacetic acid and methylenechloride. Embodiments of the present invention further embrace utilization of fibronectin at a concentration of about 1-about 50 μg/ml in deionized water or buffer solutions and where surface coating molecules on the nanofibers are laminin, poly-lysine, collagen, glycosaminoglycans, collagen, gelatin is possible while also allowing for a method to coat the surfaces of the chitosan nanofibers with glycosaminoglycans by ionically or covalently using cross-linking reagents, such as glutaraldehyde, genipin, and EDC/Sulfo-NHS.

The intercellular alignment of endothelial cells on the nanofibers can be the result of physical orientative cues from the architecture of the nanofibers. As the endothelial cells attach and migrate across the chitosan nanofibers they can cause traction by pulling on the nanofibers and communicate mechanically with neighboring cells about their spatial organization. The cells can sense the mechanical signals through their transmembrane ECM receptors such as focal contact sites.

Electrospinning is a fabrication technique by which submicron to nanometer fibers are produced as a non-woven mat from an electrostatically driven jet of polymer solution. The present invention embraces the technique of electrospinning because of its ability to produce nano-structures with a very high surface area to mass ratio. Furthermore, the fibrous structure formed by the electrospinning technique forms a network of interconnected voids that provides an environment that is similar to the in vivo ECM. A variety of synthetic or natural polymers have been used to fabricate nanofibrous scaffolds using electrospinning technique. Chitosan is used as an experimental model in certain exemplary embodiments described herein because its chemical structure is similar to the glycosaminoglycans in the extracellular matrix, and its hydrophilicity enhances its interaction with growth factors, cellular receptors, and adhesion proteins. However, the present invention is not limited to chitosan as there is no reason to believe that other commonly used fibers would not work as well.

Favored elements of the invention are best described with reference to the attached Figures.

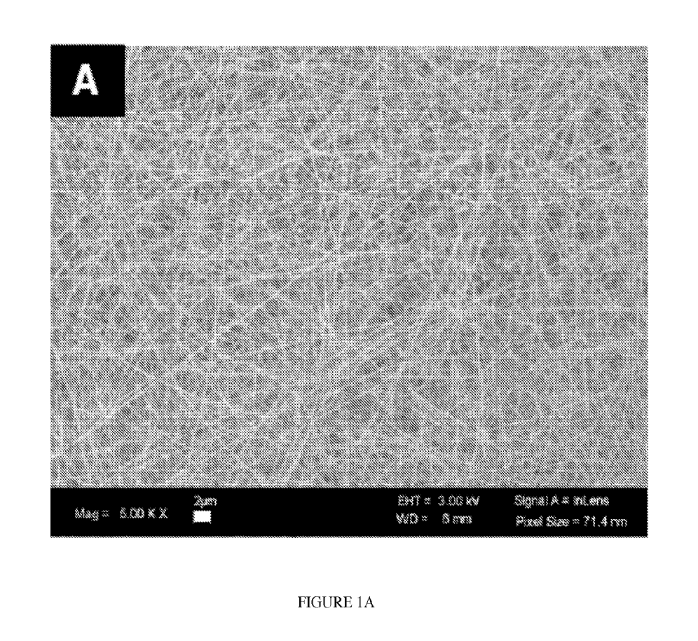

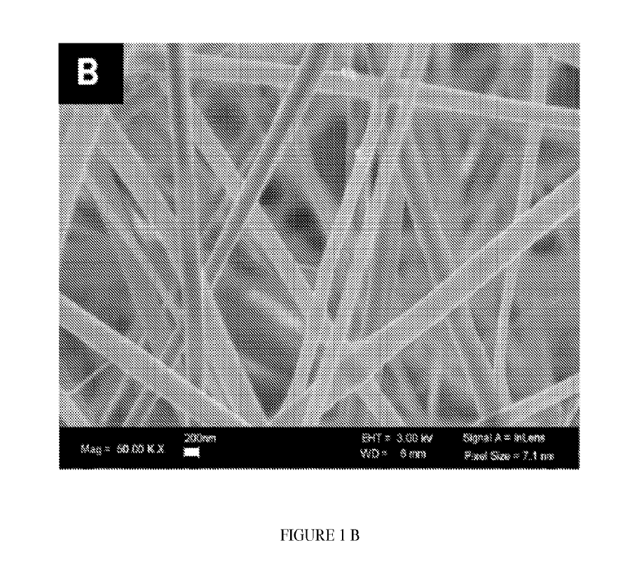

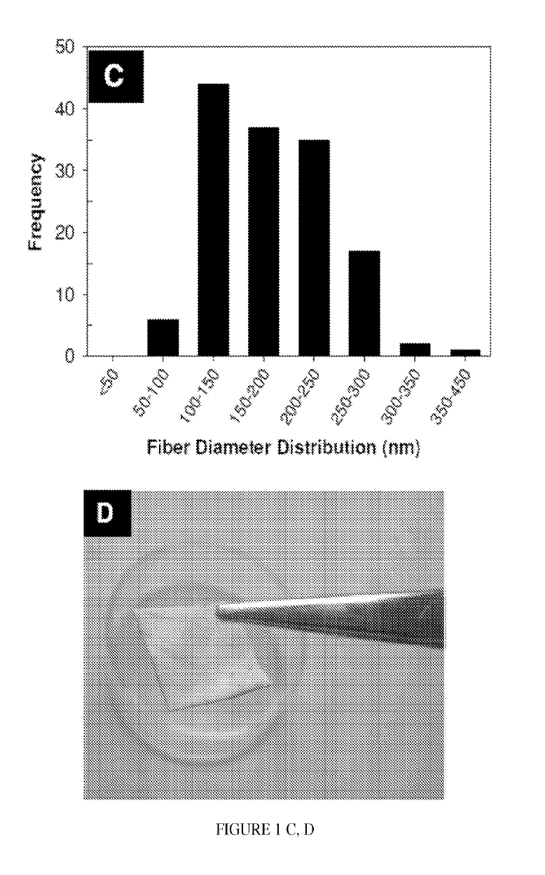

The novel woven, electrospun fibers of the present invention can be understood and described with reference to FIGS. 1A and 1B which show SEM images of the nanofibrous chitosan non-woven mats fabricated using the electrospinning technique at 5 KX and 50 KX magnifications, respectively for one embodiment of the present invention. The chitosan mats of said embodiment demonstrated homogeneous cylindrical morphology and well formed fibers with a fiber diameter ranging from about 10 nm to about 10,000 nm, and an average of 188 nm was seen in one experimental set up, as illustrated in FIG. 1C. The random orientation of the fibers produces many interconnected spaces. The fibers did not dissolve and maintained their cylindrical morphology after neutralization with ammonium hydroxide.

For one embodiment of the present invention cellular attachment to the fibers and infiltration into the interfibrous spaces was enhanced by immobilizing fibronectin onto the chitosan nanofibers by adsorption. For certain embodiments of the present invention concentrations of fibronectin can differ along the range of (0 μg/ml to approximately X0 μg/ml).

FIG. 2A illustrates an increase in observed fluorescence intensity with fibronectin concentration, which suggests that fibronectin adsorption on chitosan coated wells is dependent on the concentration of the fibronectin solution. There was a steady increase in the amount of adsorbed fibronectin as the concentration increases and the adsorption plateaus beyond 10 μg/ml. As a result, one experimental embodiment of the present invention utilized a fibronectin concentration of 10 μg/ml solution so as to allow for absorption of fibronectin on the chitosan nanofibers for improved cell adhesion from embodiments of the present invention using lower concentrations of fibronectin or no fibronectin at all.

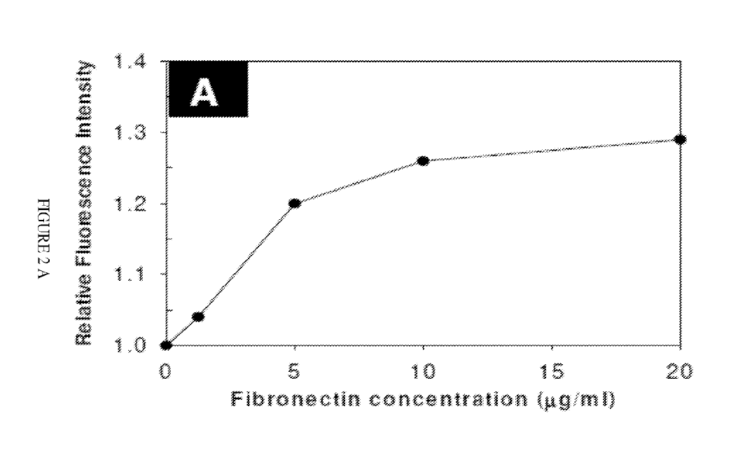

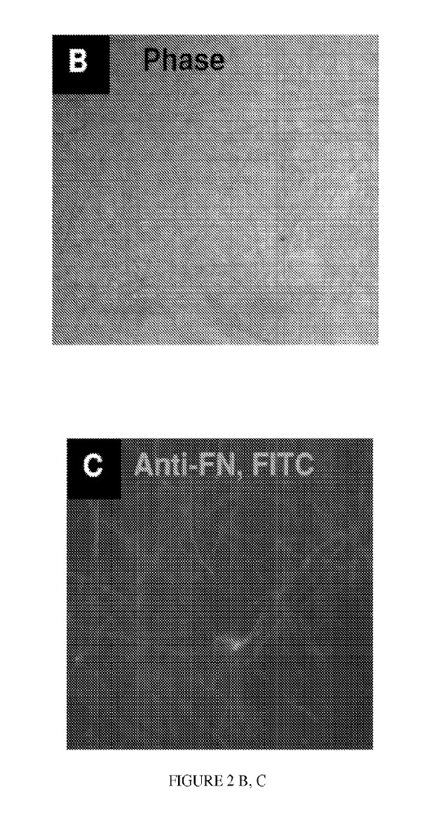

In order to investigate the effect of fibronectin immobilization on chitosan material to be utilized in cell cultures for certain embodiments of the present invention, the morphology and cytoskeletal protein distribution of endothelial cells and cardiomyocytes were monitored on 2-D chitosan films with and without fibronectin adsorption. The optical microscopy and fluorescent staining in FIGS. 3A (endothelial cells) and 3C (cardiomyocytes) depict that the cells maintain a rounded morphology, minimal vinculin (focal adhesion) expression, and a diffused F-actin cytoskeletal organization when cultured on chitosan film. In contrast, the cells cultured on fibronectin coated chitosan films demonstrated enhanced cellular spreading, significant increase in vinculin expression and a well organized fibrous F-actin cytoskeleton (FIGS. 3B and 3D). Similar results were observed in fibroblast cultures (data not shown).

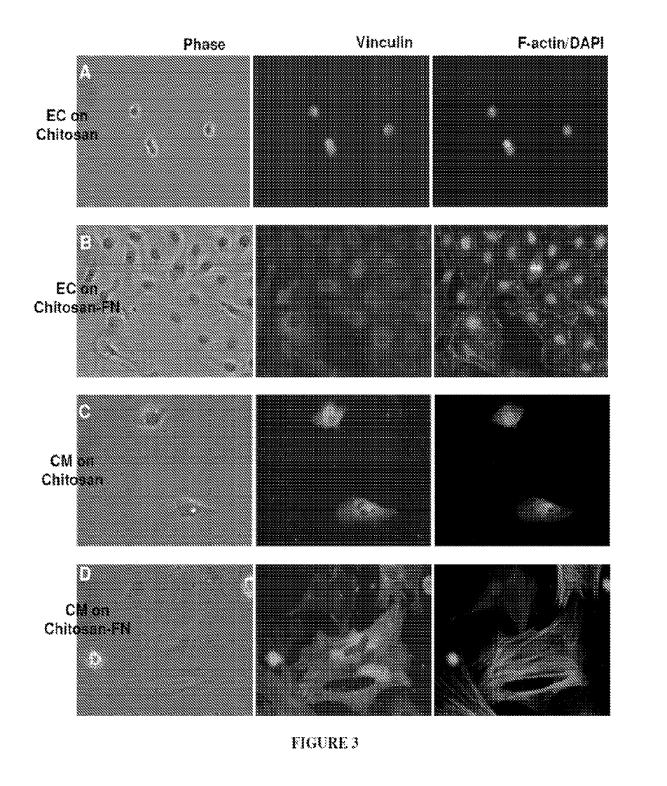

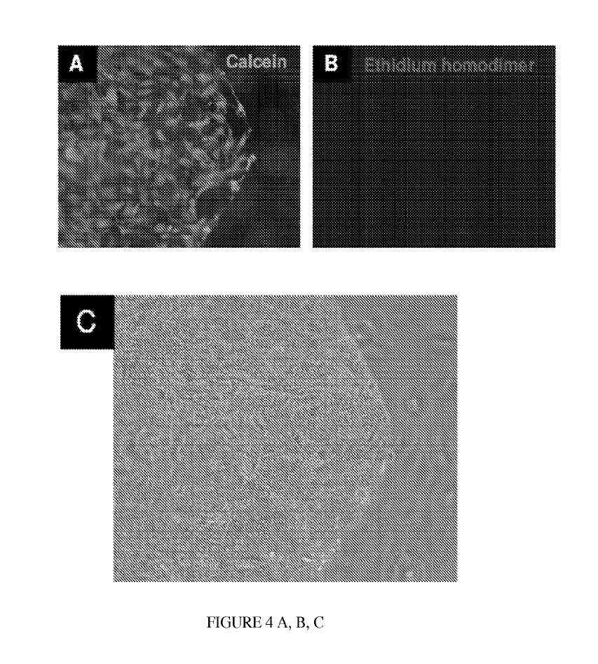

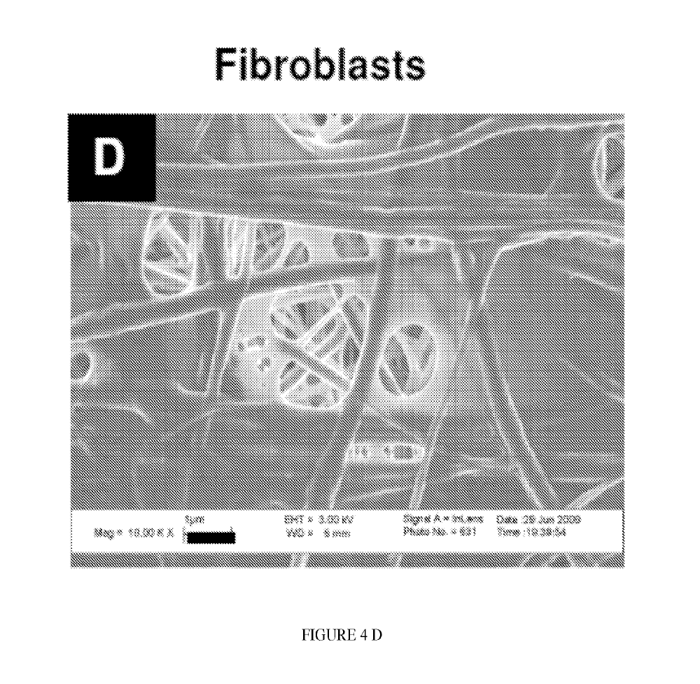

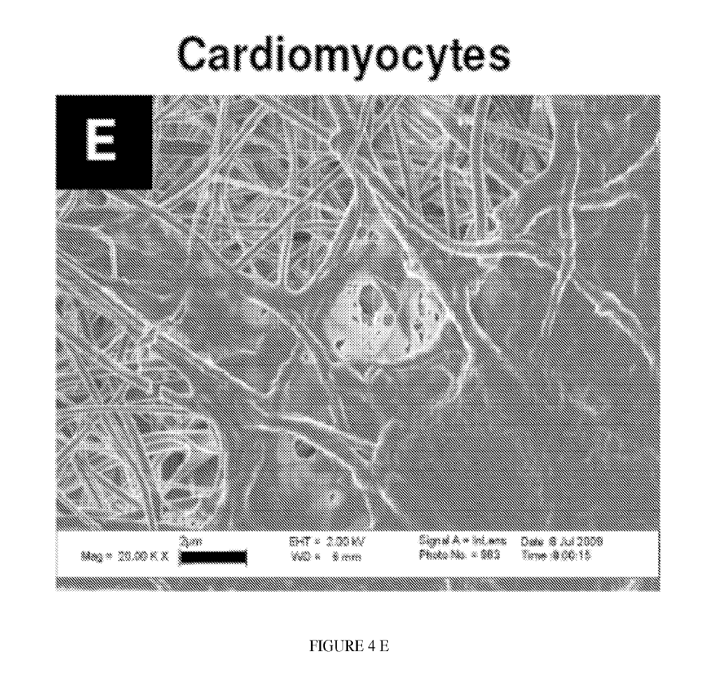

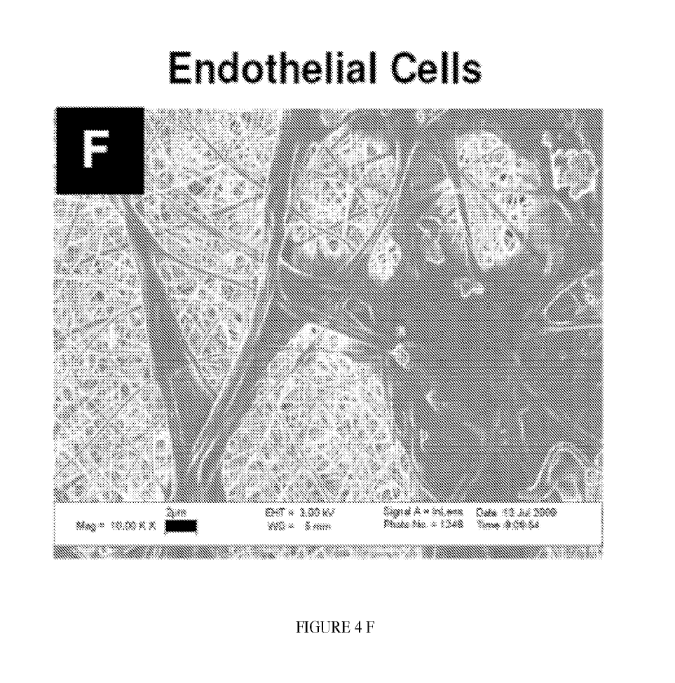

Assessment of cellular viability and morphology was performed to evaluate electrospun chitosan nanofibrous mat potential as cellular scaffolds for certain exemplary embodiments of the present invention. Fibroblasts, endothelial cells, and cardiomyocytes were seeded onto fibronectin coated chitosan mats and cultured over three weeks. FIGS. 4A-C depict the live-dead staining of fibroblasts cultured on the chitosan nanofiber scaffolds, indicating the chitosan nanofibers do not adversely affect cell viability. Some cells formed filopodia-like extensions to attach to the fibers, assisting them in spreading inside the chitosan nanofibrous scaffold (FIGS. 4D-F). In addition, the SEM images exhibit the formation of a film-like material surrounding the densely seeded areas, indicating the secretion and immobilization of cell secreted ECM components.

Chitosan is utilized in multiple present embodiments, however other fibers that could be used include, but are not limited to natural polymers such as collagen, gelatin, chitosan and synthetic polymers such as PLLA, PLGA, PCL (polycaprolactone).

Cardiomyocyte morphology and gap junction formation were monitored for one embodiment of the present invention via sarcomeric alpha-actin (SA-actin) and connexin-43 (Cx43) staining, respectively. Cardiomyocytes' SA-actin and Cx43 expression was examined on both fibronectin adsorbed chitosan films (2-D) and fibronectin adsorbed chitosan nanofibers (3-D). In each condition, cardiomyocytes were cultured in monocultures (cardiomyocytes only) and co-cultures (cardiomyocytes-fibroblasts or cardiomyocytes-endothelial cells).

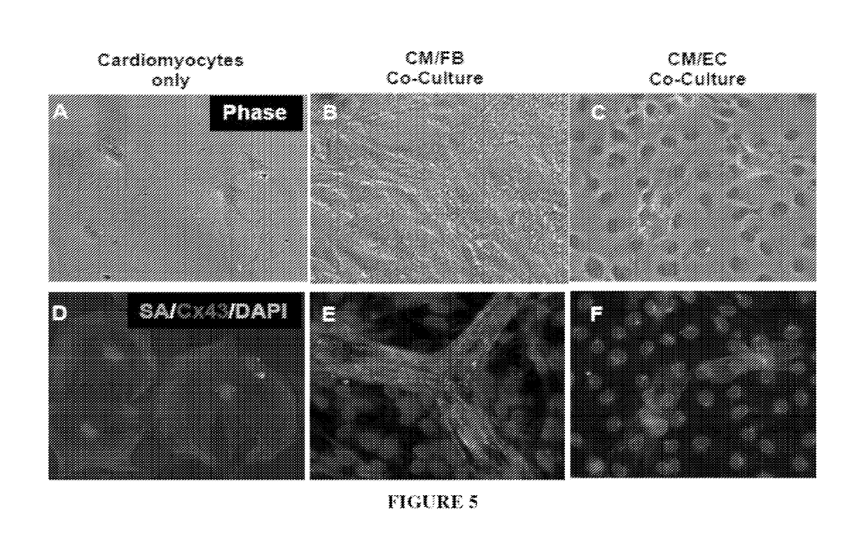

In the 2-D systems, the cardiomyocyte monoculture (FIG. 5A, D) exhibited low expression of SA-actin and the cardiomyocytes lost their structural polarity and acquired a rounded morphology. Gap junction protein Cx43 expression was minimal in the monoculture system, resulting in isolated islands of contractions (Video on file). In the fibroblasts co-culture system (FIG. 5B, E), the cardiomyocytes maintained a highly polar morphology and the SA-actin was strongly expressed along the axis of morphological polarity. In addition, Cx43 expression was the highest in the fibroblasts co-culture which enabled the cardiomyocytes to contract in a tissue-like synchronized manner. The cardiomyocytes co-cultured with endothelial cells (FIG. 5C, F) demonstrated a spherical morphology with lower levels of SA-actin and Cx43 expression than those in the fibroblasts co-culture as well as isolated contractions.

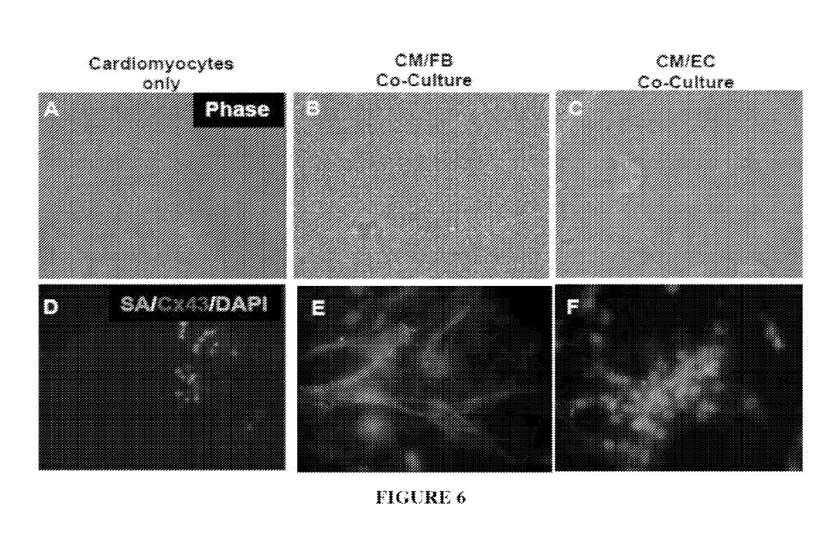

In certain exemplary embodiments of the present invention the same cardiomyocyte monoculture and co-culture studies described above were performed on 3-D chitosan nanofibers. The cardiomyocyte monoculture embodiment (FIG. 6A, D) and cardiomyocytes-endothelial cell co-culture embodiment (FIG. 6C, F) did not have any visible SA-actin or Cx43 expression. The cardiomyocyte-fibroblast co-culture embodiment resulted in elongated networks of contracting cardiomyocytes with the highest expression of SA-actin and Cx43 (FIG. 6B, E).

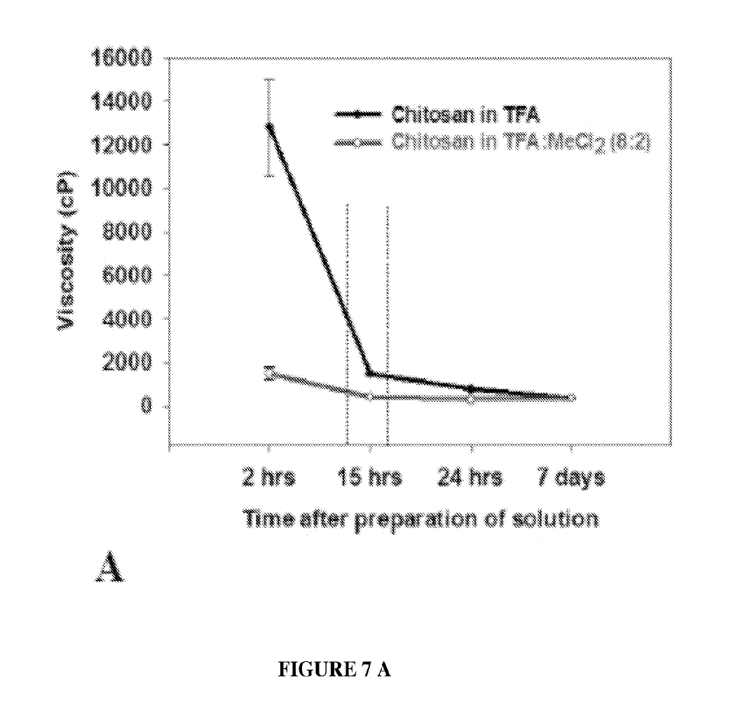

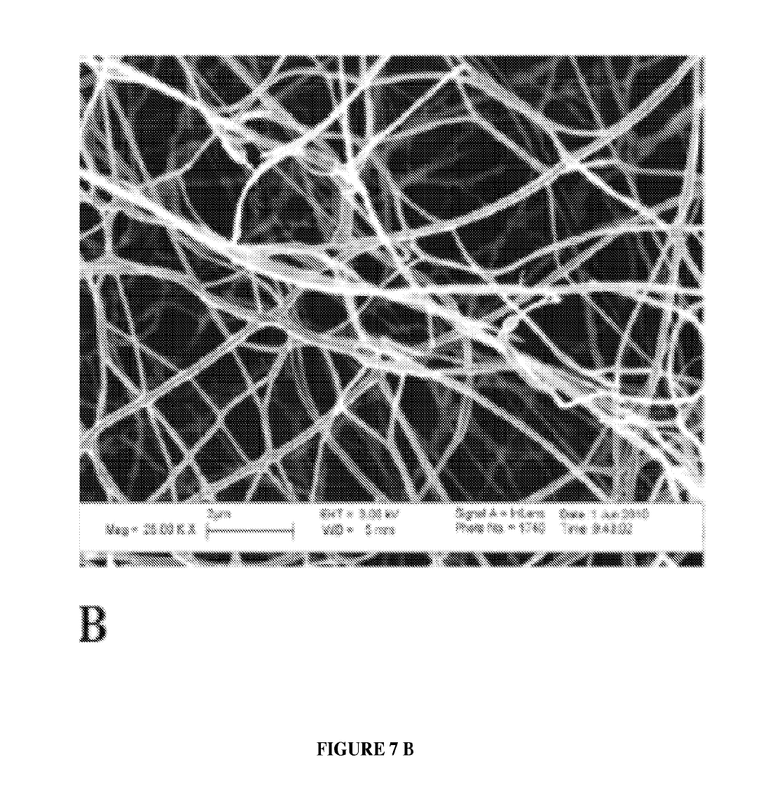

At 2 hrs after mixing the 8% (w/v) chitosan of one embodiment of the present invention in 100% TFA, the viscosity of the solution was 12,700 cP (FIG. 7A). In other embodiments the range of solution concentration is about 4-about 12%. In said embodiment, the addition of the organic solvent MeCl to the solution caused the viscosity to dramatically drop to 1,490 cP. It was observed that the viscosity of the electrospinning solution decreased as time progressed. Attempts at electrospinning the solution of said embodiment at about 2 hrs failed because the viscosity was too high for the solution to be smoothly pumped out of the needle. The optimized viscosity of the electrospinning solution that enabled the generation of smooth bead free fibers from chitosan for said embodiment was approximately 390 cP; this was achieved after approximately 12-15 hours of stirring. In other embodiments the viscosity could be anywhere from 1-about 2000 cP and stirring could be from 1-about 24 hours. The optimized chitosan fiber matrices of said embodiment had an average fiber diameter of about 188±59 nm and mat thickness approximately 150 to approximately 200 μm (FIG. 7B).

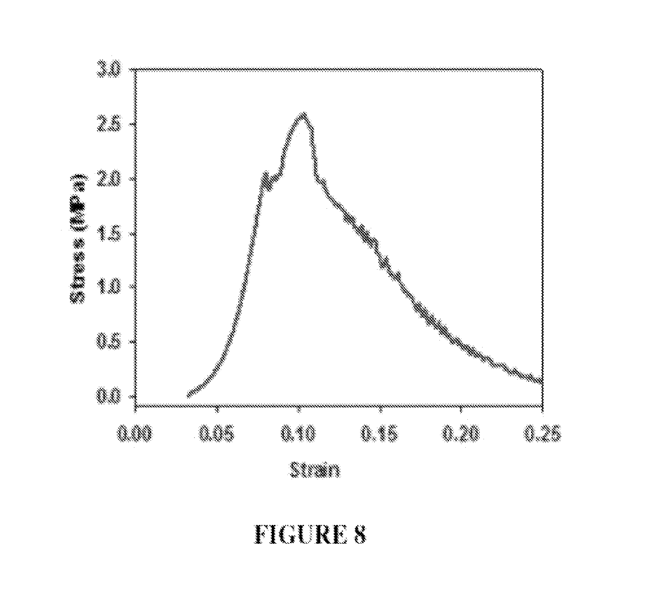

The uniaxial tensile properties of a chitosan nanofiber matrix of certain embodiments of the present invention were examined using an Instron. The stress-strain profile, seen in FIG. 8, of the matrices resulted in an evolving stress-strain modulus. During the initial phase of the tensile loading, the elastic modulus was 20.4 MPa±5 for said embodiment. Following further tensile loading onto the matrices of said embodiment, the stress-strain modulus increased significantly to 62.3 MPa±5. The matrices of said embodiment ruptured at an ultimate tensile strength of 2.20±0.37.

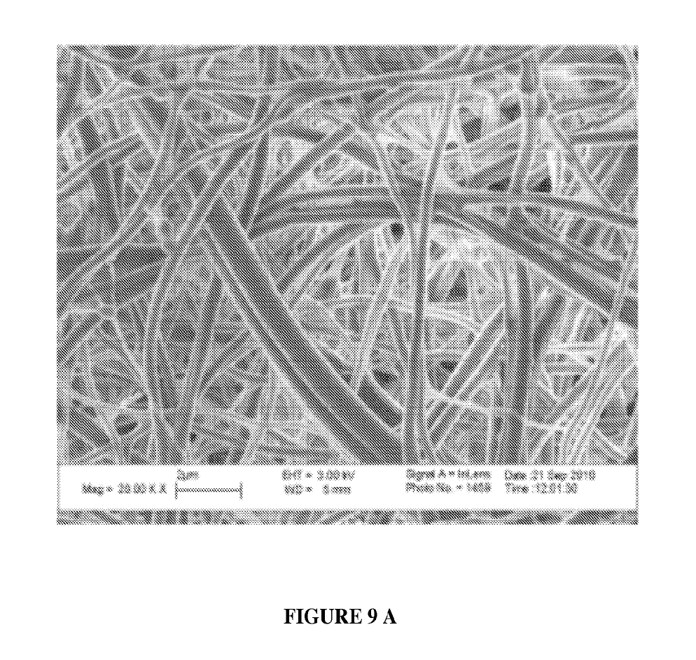

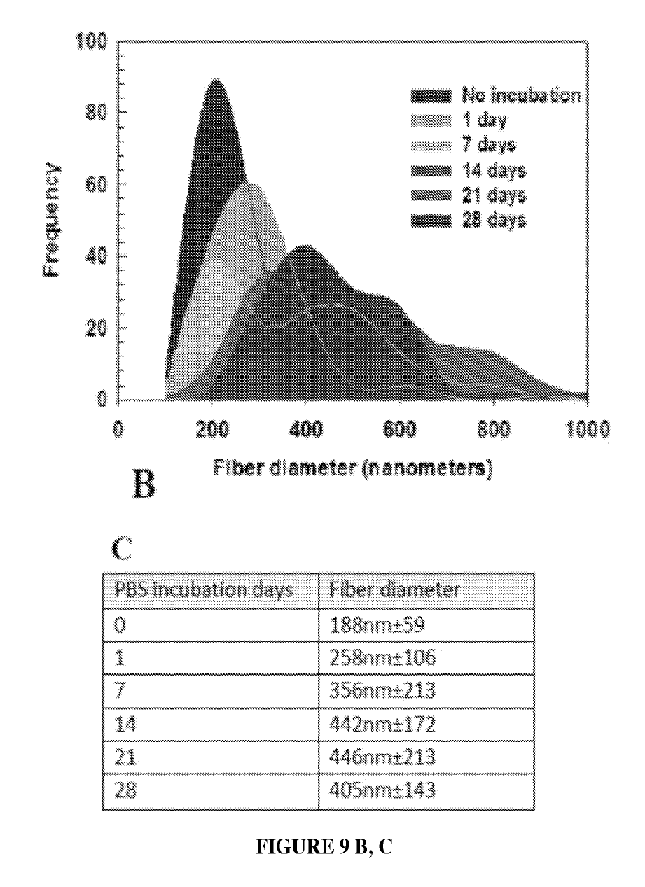

The structural stability of the fibers of said embodiments were monitored during PBS incubation over 28 days. Said fibers (0 day incubation) displayed an average diameter of 188±59 nm. FIG. 9C demonstrates that the increase in fiber average diameter and the diameter distribution range. The widening of the diameter distribution range suggests the presence of a range of swelling properties within the electrospun chitosan fibers of certain embodiments of the present invention. Hence, some fibers swell faster than others, as seen in FIG. 9A-B which demonstrates electrospun chitosan fibers of said embodiments after 28 days of PBS incubation.

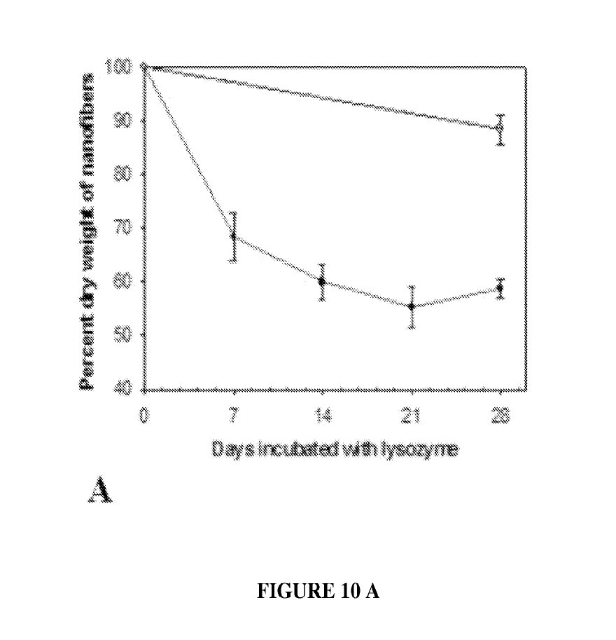

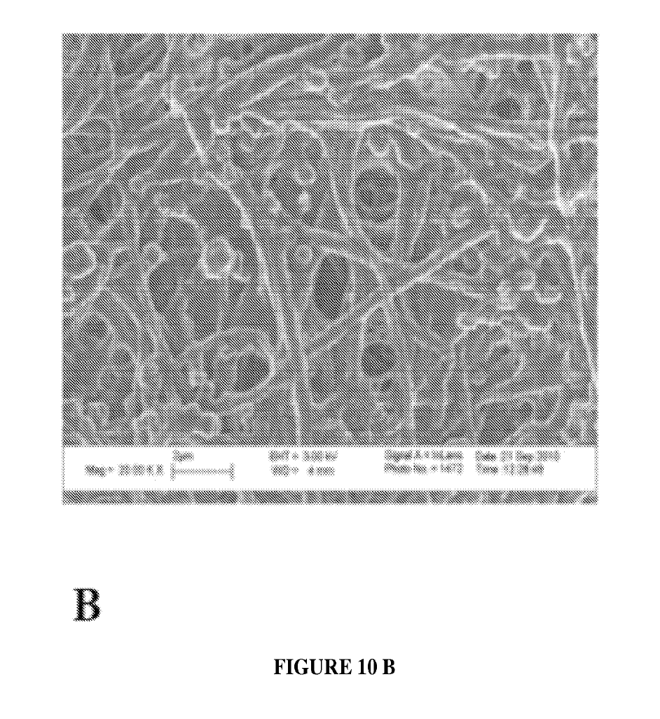

The degradation study demonstrated significant changes in the rate of degradation with time. The chitosan fiber matrices showed the highest rate of degradation during the first 7 days by displaying 30% dry weight loss. The matrices lost a further 13% dry weight during the following 21 days. The degradation profile shown using percent weight loss in FIG. 10A demonstrates that the degradation rate is initially rapid followed by a slow degradation phase. The SEM micrographs (FIG. 10B) of the chitosan matrices treated with lysozyme for 28 days depict that some of the fibers have maintained their smooth cylindrical morphology while others have degraded into textured, beaded film structures.

FTIR spectra of the chitosan film and electrospun fibers of one embodiment of the present invention demonstrate CH and CH₂ peaks at 719-793 cm⁻¹ and NH amine peak at 834 cm⁻¹ which are absent from the unprocessed chitosan powder spectra (FIG. 11A). The NH amide band was shifted downward from 1553 cm⁻¹ in the powder to 1525 cm⁻¹ in the electrospun fibers, similarly the CO amide band shifted from 1668 cm⁻¹ to 1648 cm⁻¹.

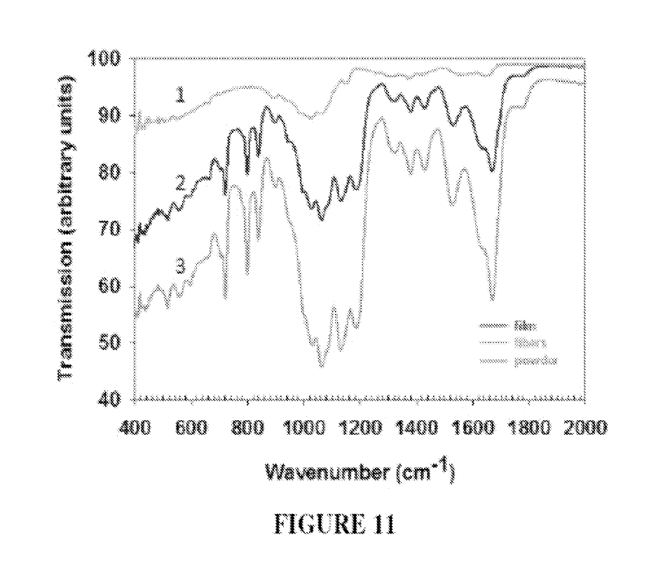

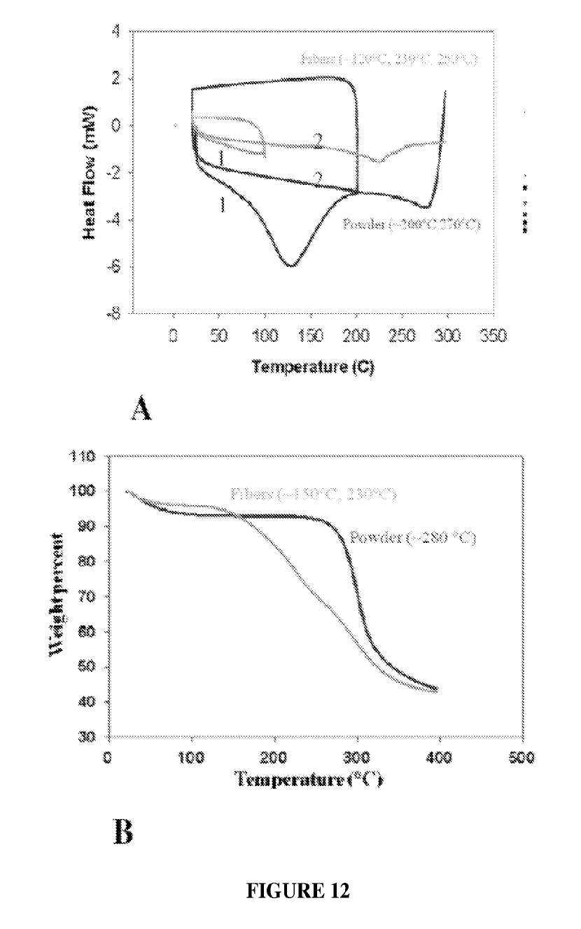

Fluctuations in heat capacity and degradation temperatures were studied using DSC and TGA. The unprocessed chitosan powder and the electrospun chitosan fibers of certain embodiments of the present invention were compared. DSC profiles demonstrated broad endothermic events with peak maxima at approximately 90° C. for the electrospun chitosan fibers and 140° C. for the chitosan powder during the first scan. The second DSC scan illustrated a change in heat capacity at about 120° C., and a broad endothermic event at about 230° C. followed by another event at about 250° C. The chitosan powder's second DSC profile demonstrated a change in heat capacity at 200° C. and an endothermic event at 270° C. (FIG. 12A).

TGA showed an initial weight loss that plateaus in both the electrospun chitosan fibers and powder. Further weight loss of the electrospun chitosan fibers of said embodiments started at a temperature of ˜150° C. as compared to the powder which started at ˜280° C. (FIG. 12B)

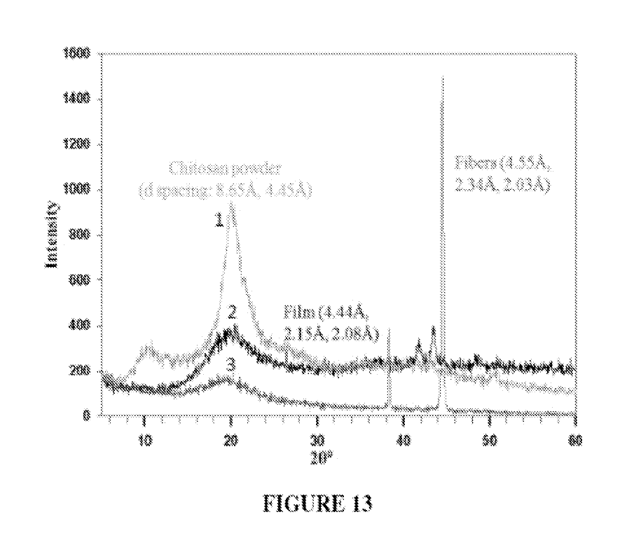

As depicted in FIG. 13, the unprocessed chitosan powder had two broad peaks at 2-theta=10.2° and 19.9°, in addition to a broad background halo. The cast film from the chitosan electrospinning solution of one embodiment of the present invention resulted in one broad peak at 2-theta=19.9°. Said embodiment XRD pattern resulted in the lowest intensity peak at 2-theta=19.9, two very sharp and intense peaks at 2-theta=38.3° and 44.5° and the lowest amount of background halo.

In one embodiment of the present invention an 8% (w/v) chitosan solution was prepared by dissolving chitosan (medium molecular weight ˜200K, 75-85% deacetylation; Sigma) in Trifluoroacetic acid (TFA; Fisher Chemicals). The solution of said embodiment was stirred overnight at 40° C. Methylene Chloride (MeCl; Fisher Chemicals) was added to form a final volume to volume ratio of 80:20 (TFA:MeCl) for said embodiment. In other embodiments the range a final volume to volume ratio of about 95:5 to about 65:35 (TFA:MeCl). The chitosan solution of said embodiment was fed into a 10 ml disposable syringe fitted with an 18 gauge needle. In other embodiments of the present invention a syringe of about 1 ml-10000 ml could be utilized as well as a needle gauge of about 10 to about 35. A DC voltage of 30 kV was applied to the needle and the planar collector was placed 30 cm from the needle for said embodiment. The polymer solution of this particular exemplary embodiment was pumped at a rate of 2 ml/hr and the process was performed at room temperature and atmospheric humidity of about 40- to about 50%. For said embodiment, following vacuum drying at room temperature, the chitosan nanofibers were cut into pieces to fit into 35-mm dish and neutralized with 15N ammonium hydroxide: 100% ethanol (1:1 v/v ratio) for 30 mins. The chitosan nanofibers were then washed with distilled water 3 times for 15 minutes each time. The chitosan nanofibers were then sterilized under a UV lamp for 20 minutes.

In order to evaluate fibronectin adsorption on chitosan, 24-well tissue culture dishes were coated with 1% chitosan and dried for 1 hr before neutralization with 0.2 M ammonium hydroxide. Fibronectin (Sigma) solutions of different concentration (0 μg/ml, 0.6 μg/ml, 1.2 μg/ml, 5 μg/ml, 10 μg/ml, and 20 μg/ml in deionized water) were added into the dishes and incubated for 1 hour. The amount of adsorbed fibronectin was characterized by fluorescent staining using anti-fibronectin antibody (Sigma).

In one embodiment of the present invention, primary ventricular cardiomyocytes were isolated from 1-day-old neonatal Wistar rats (Charles River Laboratories, MA) using a collagenase procedure as described previously (Aoki et al. 1998) and cultured on 0.1% gelatin-coated dishes. Cardiomyocyte culture medium consists of DMEM (Gibco, Gaithersburgh, Md.) with 10% FBS (Biowest, Miami, Fla.), 2 mM L-glutamine (Gibco), insuliriltransferrin/selenious acid (ITS; 5 μg/mL, 5 μg/mL, 5 ng/mL, respectively) (Invitrogen), and 2% penicillin/streptomycin (Gibco). For said embodiment, Murine 3T3-J2 fibroblasts (purchased from Howard Green, Harvard Medical School, Boston, Mass.), were maintained in 60-mm tissue culture dishes in DMEM plus 10% FBS and 2% penicillin and streptomycin. For said embodiment, rat heart microvascular endothelial cells (MVEC; purchased from VEC Technologies, Rensselaer, N.Y.), were maintained in DMEM supplemented with 10% FBS, 2 mM L-glutamine, ITS, 2% penicillin/streptomycin, and 10 ng/mL vascular endothelial growth factor (VEGF, R & D Systems, Minneapolis, Minn.). Culture medium was changed every three days for said embodiment of the present invention.

For the 2-D cell cultures, the chitosan or chitosan/FN films were prepared in 24-well tissue culture dishes. Cells were seeded at a density of ˜25,000 cells/cm² on the films in 0.5 mL of culture medium. For 3-D cell cultures of one embodiment of the present invention, the nanofibers were treated with fibronectin solution (10 μg/ml) for 1 hour to adsorb fibronectin on the fibers. The nanofibers were then soaked in culture medium for about 5 minutes prior to cell seeding. The suspended cells (fibroblasts, cardiomyocytes, or endothelial cells) in culture medium were directly seeded at a density of ˜300,000 cells/cm² on separate chitosan nanofibrous mats with 300 μl of medium and incubated for about 1 hour before adding further medium to allow for better cell entrapment and attachment. For the co-culture studies (cardiomyocytes-fibroblasts or cardiomyocytes-endothelial cells co-culture) in 2-D and 3-D culture systems, cells were seeded at the ratio of 1 to 1, giving a total initial cell number (cardiomyocytes+fibroblasts or cardiomyocytes+endothelial cells) of ˜50,000 cells/cm² for 2-D and ˜600,000 cells/cm² for 3-D cultures. The mats were incubated in a 10% CO₂, 37° C. incubator and the medium was changed every 3 days.

In one embodiment of the present invention samples were washed with PBS and fixed with 4% paraformaldehyde for 20 minutes at room temperature. After washing with PBS, said embodiment was permeabilized in 0.2% Triton X-100 in PBS for 10 minutes. The present embodiment was then washed with PBS and incubated in blocking buffer (PBS/10% FBS/1% BSA) for 30 minutes. The primary antibody was then added to said embodiment and incubated for 60 minutes at room temperature. Samples of said embodiment were then washed with PBS and then incubated with the secondary antibody for 60 minutes before being washed and examined with fluorescence microscopy (Nikon). The primary antibodies used in said embodiment were mouse anti-vinculin (Millipore, 1:200 dilution), mouse anti-α-sarcomeric actin (Invitrogen, 1:50 dilution), and rabbit anti-connexin 43 (Sigma, 1:1000 dilution). The secondary antibodies used in said embodiment were donkey anti-mouse IgG, alexa fluor 488 and donkey anti-rabbit IgG, alexa fluor 594 (Invitrogen). For the distribution of actin microfilaments of said embodiment, cells were stained by incubating fixed and permeabilized cultures with about 0.1 μg/mL rhodamine phalloidin (Sigma) for 30 min. In some cases, cells were counterstained with 4, 6-diamidino-2-phenylindole (DAPI; Invitrogen) for nuclear staining. In order to verify the adsorption of fibronectin onto chitosan of said embodiment, the samples were stained for 1 hr at room temperature with rabbit anti-fibronectin. After washing twice with PBS, the samples of said embodiment were incubated with alexa fluor 488 conjugated anti-rabbit IgG, and washed twice with PBS. Cell viability on the nanofiber scaffolds was examined by a live/dead viability/cytotoxicity kit (Invitrogen).

The cell seeded nanofibers scaffolds of certain embodiments of the present invention were examined with SEM. In one embodiment, the samples were fixed with 2.5% glutaraldehyde for 24 hours at 4° C. Said samples were then washed with PBS and serially dehydrated with 50%, 70%, and 100% ethanol for 15 minutes each. This was done to allow gradual dehydration of the cells preventing loss of cellular structural integrity. Said samples were then vacuum-dried for about 6 hours. Said samples were coated with carbon and observed with SEM.

Nikon Imaging Solutions (NIS)-elements imaging software program (v. 3-448) was used to measure the diameter distribution of the chitosan fibers. The measurements were done from five different SEM images on different regions of each sample. A total of ˜150 measurements were analyzed for the fiber diameter. To quantify the amount of adsorbed fibronectin on chitosan surfaces, average fluorescence intensity were measured and analyzed after staining with anti-fibronectin, FITC. The experiments were performed at least twice in duplicate.

For certain embodiments of the present invention the stress-strain modulus and ultimate tensile strength were determined using an Instron uniaxial tensile testing equipment model 3343. The chitosan nanofiber matrices of said embodiments were prepared into rectangular strips (60×20 mm) while the relative humidity was approximately 20-approximately 30%, gauge was set to 20 mm and cross head speed of 10 mm/min was used. All the strips were tested until complete rupture with a 100N load cell at room temperature (25-30° C.).

In one embodiment of the present invention the chitosan nanofiber matrices were prepared into squares (20×20×0.15 mm3) and neutralized as mentioned above. The degradation of the chitosan nanofibers of said embodiment was assessed by incubating them in egg-white lysozyme (MP Biomedicals LLC, CAT#100834) dissolved at 4 mg/ml in PBS, pH 7.2, at 37° C. and 10% CO₂. At specific time intervals (days 7, 14, 21, 28) the matrices were removed from the lysozyme solution, washed with water, vacuum dried for 24 hours, and weighed. The degradation profile for said embodiment was illustrated using the weight loss percentage of the dried sample before and after degradation.

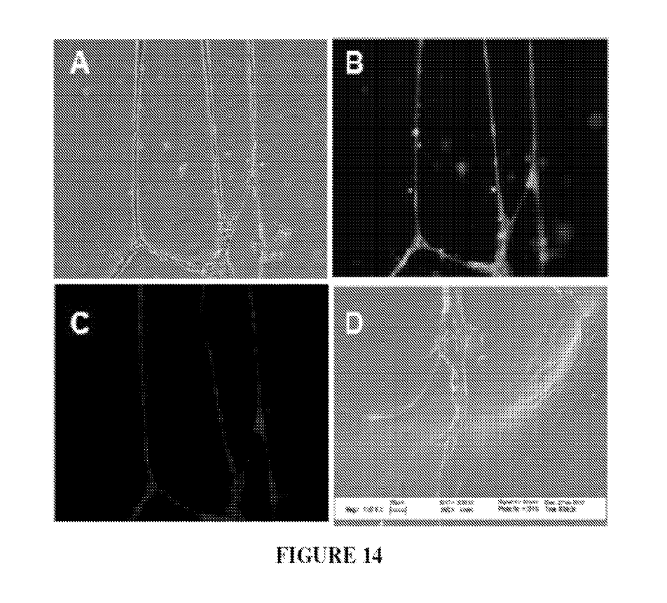

Again, in order to test for vascular network-like formation of endothelial cells, we assayed tube formation in 2-D culture using Matrigel. As shown in FIG. 14, most of the cells in this exemplary embodiment formed vascular network-like structures on Matrigel, indicating their capability to form capillary network.

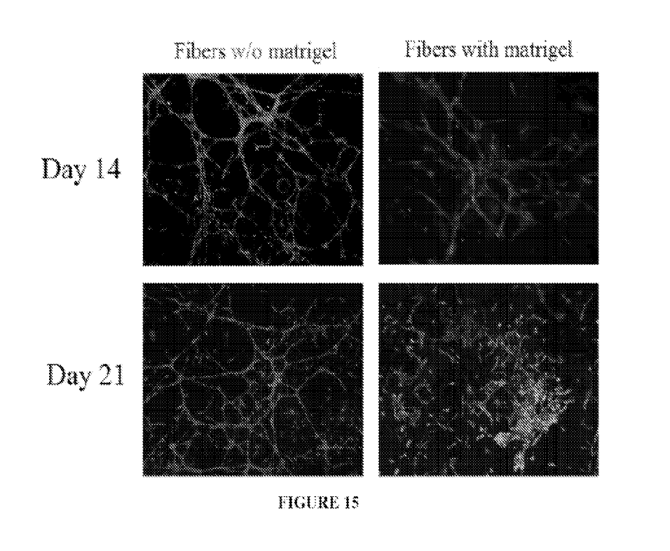

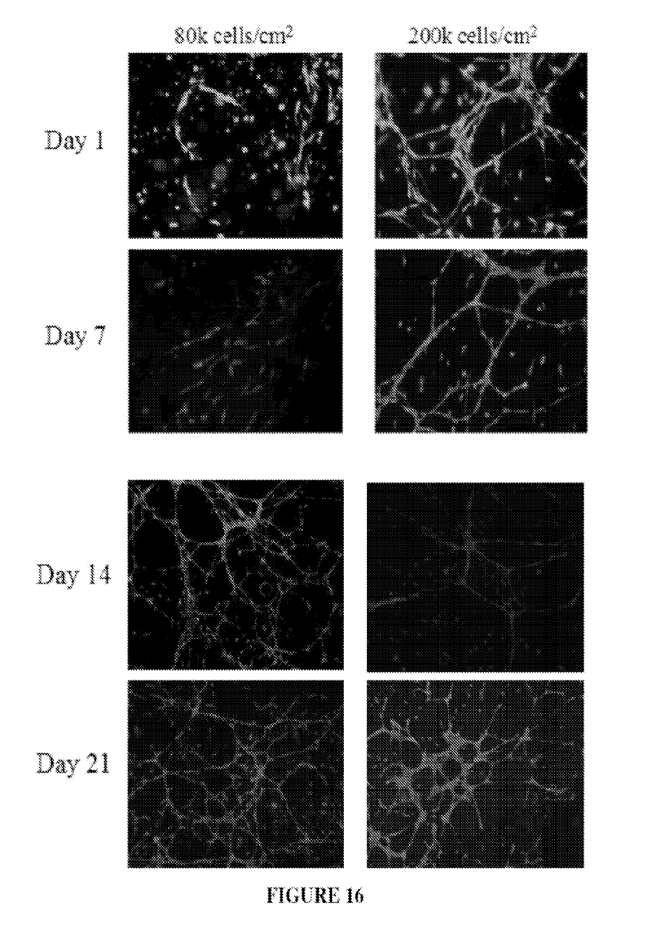

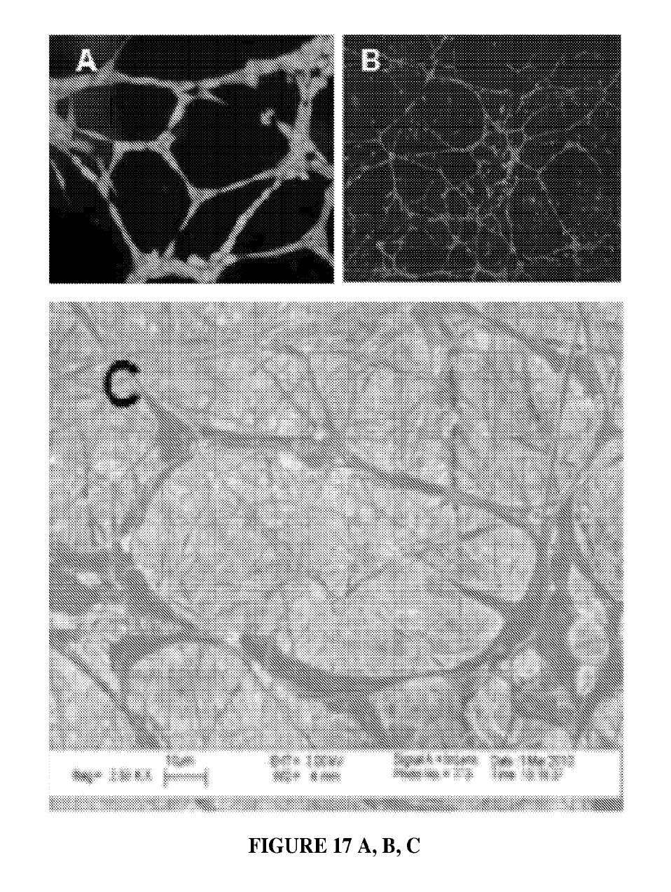

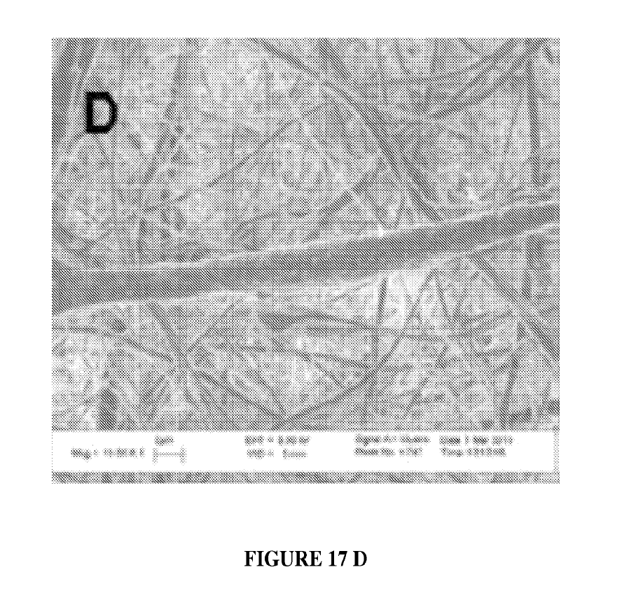

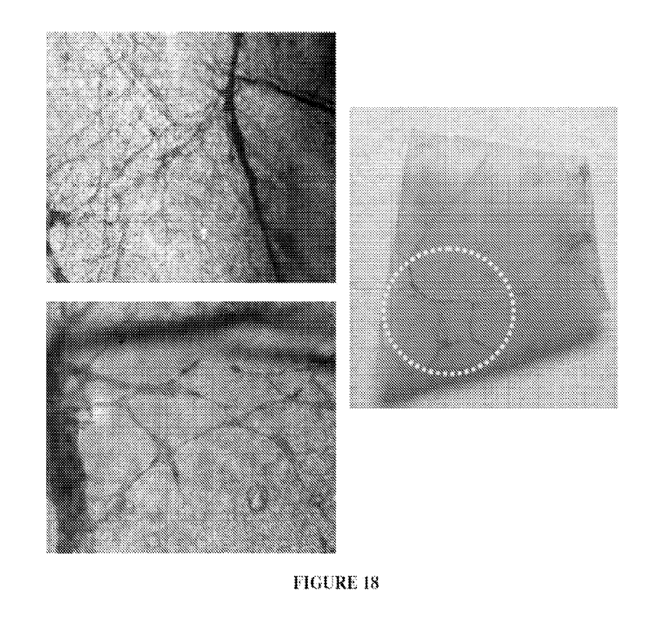

In addition, tube formation was also investigated on the 3D chitosan nanofibers with and without the use of Matrigel. Results in 3D nanofiber scaffolds indicate that endothelial cells seeded within nanoscaffolds without the use of the animal-derived Matrigel exhibit capillary-like tube formation on the nanofiber structure (FIG. 15) for certain embodiments of the present invention. Similar tube formation was observed in embodiments employing Matrigel-coated nanofiber scaffolds. Results clearly demonstrate that endothelial cells (LSEC) have migrated across the nanofibers and have aligned themselves linearly to form interconnected network tubes (FIG. 16-18). This capability of endothelial cells to form network tube-like structures in a 3-D nanofibrous scaffold is a novel finding that has not been previously reported.

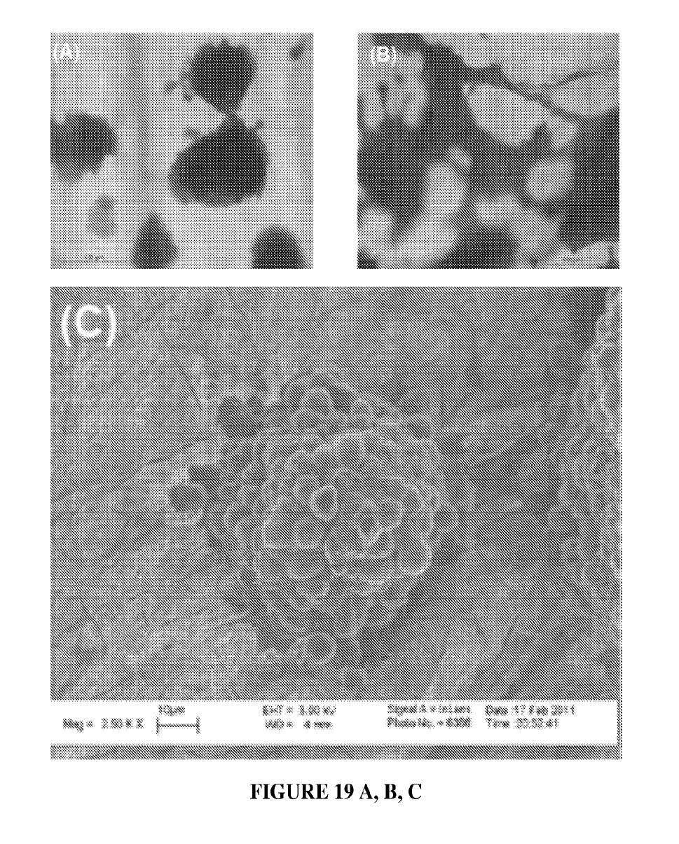

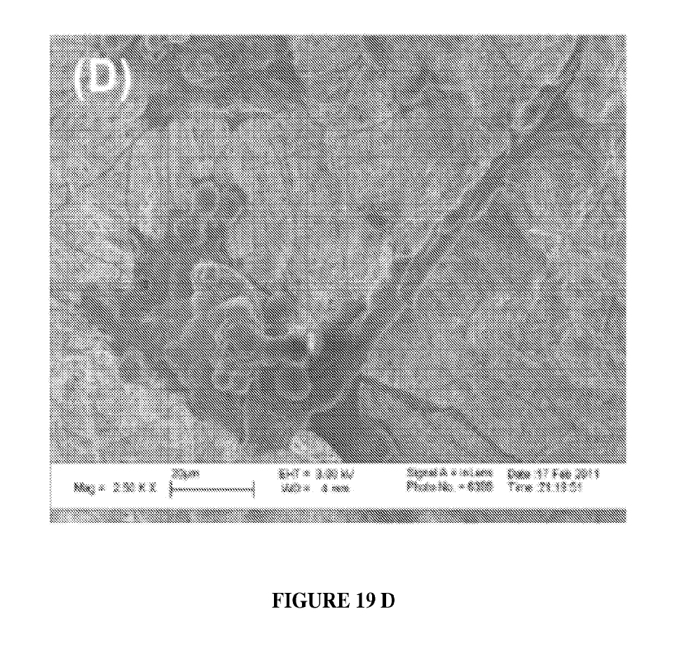

Further embodiments of the present invention show the utilization of human liver cells (hepatocytes, e.g. human HepG2) on the 3D nanofibers. FIG. 19 shows embodiments of the present invention utilizing human HepG2 cells cultured alone (FIG. 19A, C) and co-cultured with endothelial cells (FIG. 19 B,D) on the 3D chitosan nanofiber. As shown, capillary-like tube network was formed in HepG2/endothelial cell co-culture embodiment.

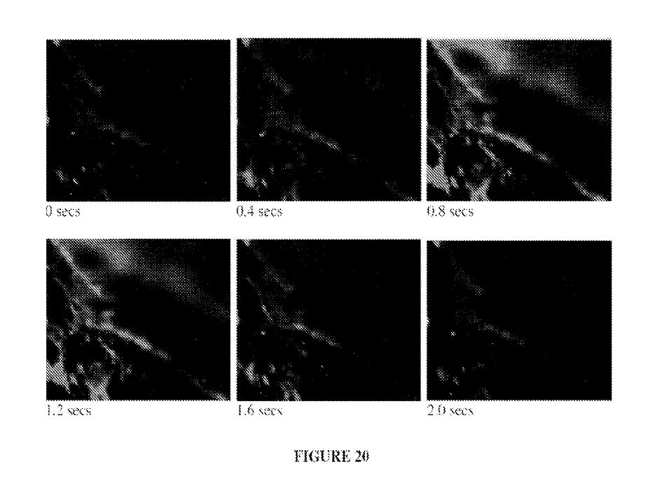

The ability of the tri-culture system to prolong cardiomyocyte viability and induce inter-cellular alignment, tubular morphogenesis, and the conduction of the electrical wave has also been demonstrated. In order to investigate calcium ion flow in cardiomyocytes in 3-D tri-culture system, in one embodiment of the present invention, cardiomyocytes were co-cultured with endothelial cells and fibroblasts in electrospun chitosan nanofibers for 7 days and loaded with the green-fluorescent calcium indicator, fluo-4 AM. Images of loaded cells were obtained with a Nikon Element fluorescence imaging system at a constant frame rate of ˜8 frames/second. Pseudo-color analysis of the transmembrane transient calcium ion flow for said embodiment shows that the cardiomyocytes have migrated and attained inter-cellular alignment to form interconnected tubular morphologies (FIG. 20). In addition, the intensity pattern of the calcium wave across the electrospun chitosan mat of said embodiment suggests that the triculture system has induced electrical wave conduction across distances of around 100 μm.

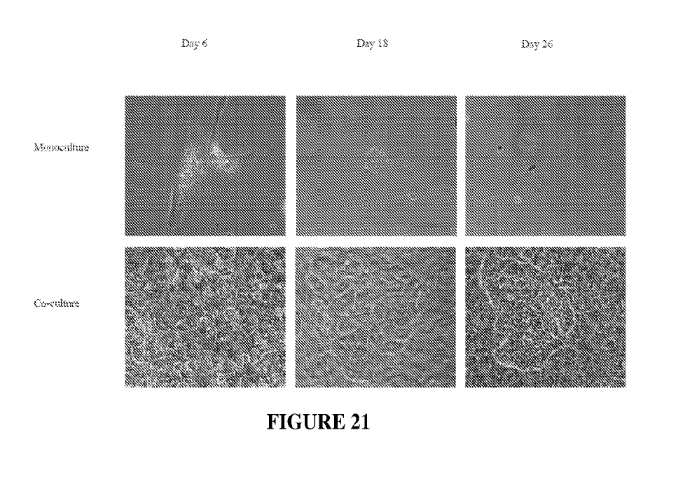

Hepatocytes were cultured alone and co-cultured with fibroblasts on fibronectin coated surfaces. Phase contrast images of the morphological characteristics of hepatocytes in monoculture and co-culture on Day 6, Day 18 and Day 26 are shown in FIG. 21, Bar: 100μ.

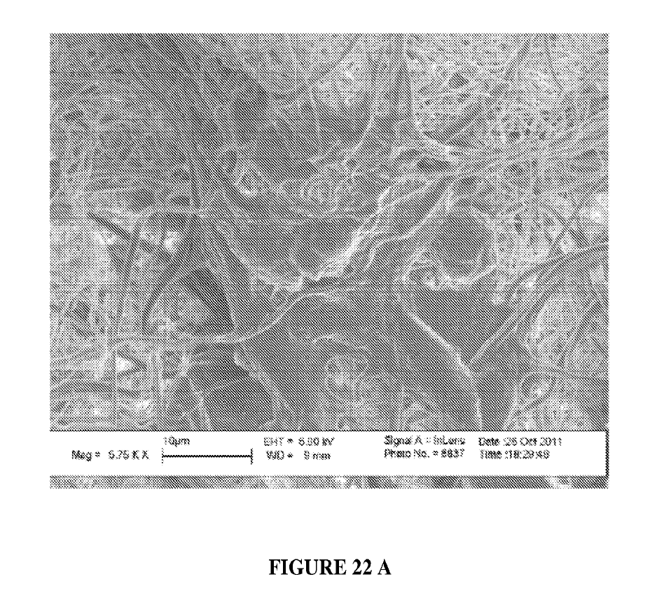

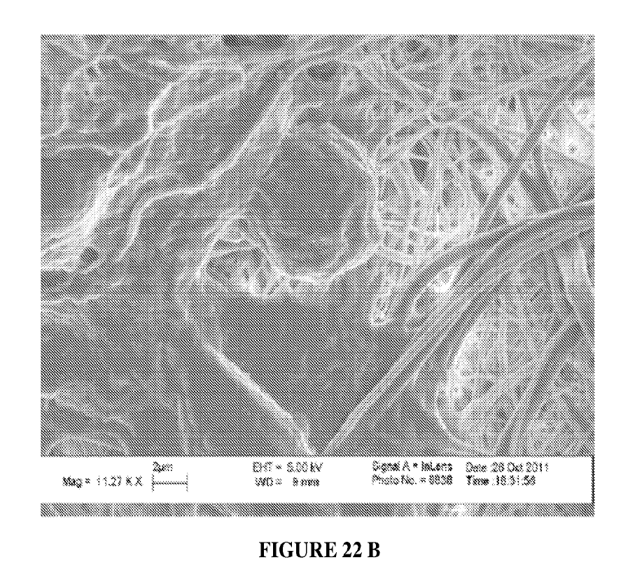

Interactions between hepatocytes and fibroblast of co-cultured 3-D liver model are clearly depicted in 3-D-SEM images of morphological characteristics of co-cultured 3-D liver model for Day 14 as shown in FIG. 22.

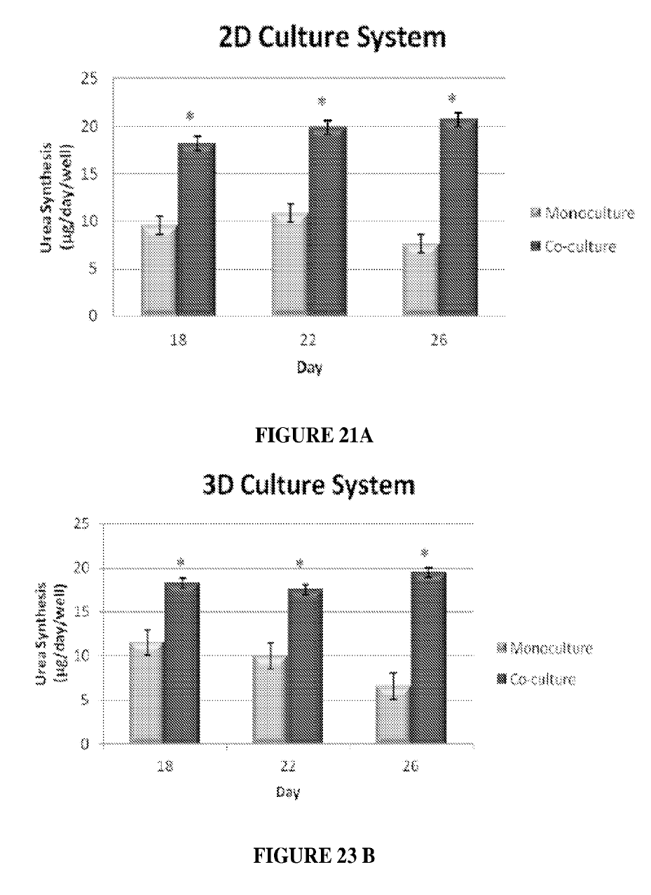

FIG. 23a shows urea synthesis in 2D culture system with urea production of (i) monoculture of primary rat hepatocyte (ii) co-culture of primary rat hepatocytes and fibroblasts cultured on fibronectin coated dish shown on Day 18, Day 22 and Day 26. Each data point showed is the mean (n=6).

Similarly, FIG. 23b shows urea synthesis in 3D culture system with urea production of (i) monoculture of primary rat hepatocyte (ii) co-culture of primary rat hepatocytes and fibroblasts cultured on fibronectin coated-chitosan nanofibers on Day 18, Day 22 and Day 26. Each data point showed is the mean (n=6). A student t-test showed that p<0.05 for all cases.

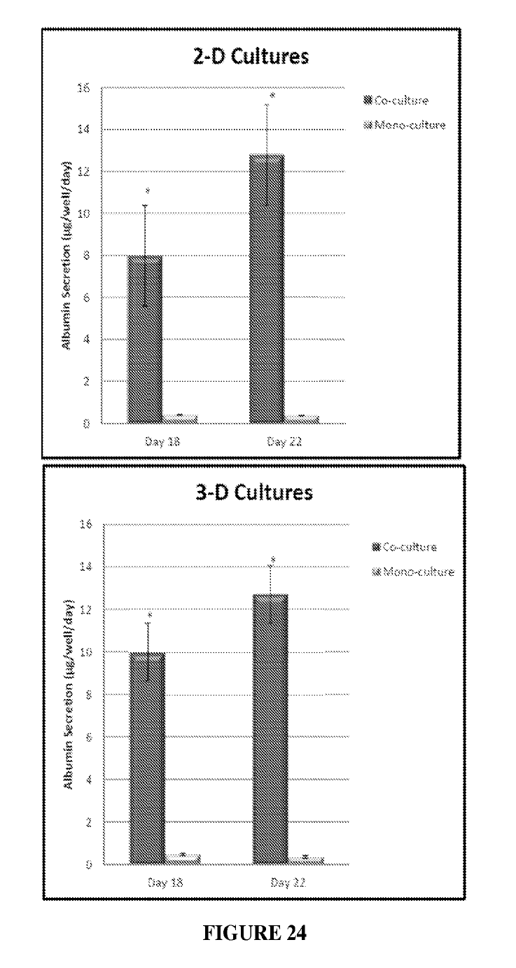

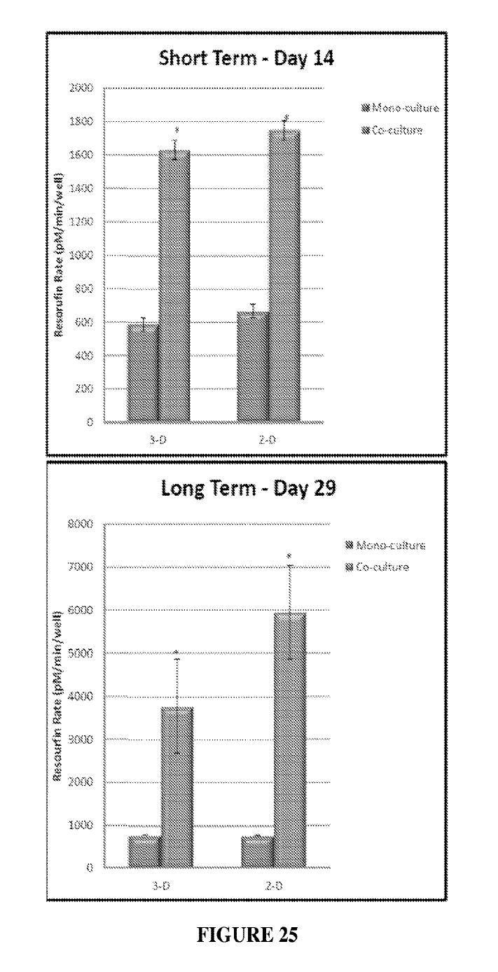

Production of albumin is one of the main synthetic functions of hepatocytes, as it constitutes up to 25% of total proteins synthesized in the liver. The co-cultures in both 2-D and 3-D were stained for albumin on Day 14 and images were obtained as shown in FIG. 24. The DAPI image shows a large number of cells however, only the hepatocytes colonies are stained in red, indicating albumin secretion. Thus, it differentiates the hepatocyte colonies from fibroblasts in the co-cultures. Albumin secretion on Day 4, 8, 12, 18 and 22 are shown in FIGS. 25a and b. The production of albumin for monocultures reached a peak on Day 4 and then slowly decreased in both 2-D and 3-D cultures. However, in the case of co-cultures, it was shown that there is gradually increase in albumin secretion as the days progressed. Student t-test indicates that there is no statistical significance between the albumin concentration on Day 4 and Day 8 between monocultures and co-cultures in 2-D scaffolds.

CYP 450 enzymes family is essential for detoxification and metabolism of drugs in the body. There are about 50 enzymes altogether out of which 5 are responsible for 90% of drug metabolism. EROD assay was conducted to determine the CYP450 1A enzyme induction by the hepatocyte cells cultured in monoculture and co-culture.

Dealkylation of 7-ethoxyresorufin by adult rat hepatocytes are shown in FIG. 26. In monoculture the formation of resorufin by Day 14 is markedly low compared to that found in co-cultures in both 2-D and 3-D cultures. CYP450A1 induction was also markedly elevated in co-cultured cells on nanofibers in long term (day 29) cultures. The values are expressed as means±standard deviations.

High levels of cytochrome in 2-D cultures may be attributed to the large interindividual variations that exist between individual cytochrome P450 enzymes due to phenotypic differences of fenetic polymorphisms. Cells in 2-D prevalently proliferate and de-differentiate, which leads to morphological and functional differences from that of original tissues. The 3-D culture system is closer to the environment found in in vivo and thus, it may be assumed that the cytochrome levels observed here, may be closer to that found in nature.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Methods and Examples

### Preparation of 1% Gelatin

For the preparation of 1% gelatin, 0.5 g of gelatin powder was dissolved in 49.5 ml of autoclaved distilled water at 50° C. The solution is filtered using a vacuum filter and the solution is stored in 20° C. for later use.

### Preparation of 1% Chitosan

For the preparation of 1% chitosan, 0.5 g of chitosan powder was mixed with 49.5 ml of distilled H2O. The homogeneous chitosan solution was either used immediately or was stored in 20° C. for later use.

### Preparation of 2-D Chitosan-Fibronectin Scaffold

Tissue culture dishes were coated with 300 μL of 1% chitosan solution for overnight incubation at room temperature. A mixture of NH₄OH:C2H5OH (1:1) was added to each well plate to neutralize the chitosan coating for 5 min at room temperature in the sterilized culture hood. The dishes were than washed with sterile dH2O thrice for 10 minutes each. Sterile fibronectin (10 μg/ml) was added to the plates and incubated for atleast an hour. The fibronectin was aspirated and the cells were seeded.

### Electrospinning of Chitosan

An 8% (w/v) chitosan solution was prepared by dissolving chitosan (medium molecular weight ˜200K, 75-85% deacetylation; Sigma) in Trifluoroacetic acid (TFA; Fisher Chemicals). The solution was stirred overnight at 40° C. Methylene Chloride (MeCl; Fisher Chemicals) was added to form a final volume to volume ratio of 80:20 (TFA:MeCl). The chitosan solution was fed into a 10 ml disposable syringe fitted with an 18 gauge needle. A DC voltage of 30 kV was applied to the needle and the planar collector was placed 30 cm from the needle. The polymer solution was pumped at a rate of 2 ml/hr and the process was performed at room temperature and atmospheric humidity of 40-50%. Following vacuum drying at room temperature, the chitosan nanofibers were cut into pieces to fit into 35-mm dish and neutralized with 15N ammonium hydroxide: 100% ethanol (1:1 v/v ratio) for 30 mins. The chitosan nanofibers were then washed with distilled water 3 times for 15 minutes each time. The chitosan nanofibers were then sterilized under a UV lamp for 20 minutes.

### Fibronectin Adsorption on Chitosan Nanofiber

In order to evaluate fibronectin adsorption on chitosan, 24-well tissue culture dishes were coated with 1% chitosan and dried for 1 hr before neutralization with 0.2 M ammonium hydroxide. Fibronectin (Sigma) solutions of different concentration (0 μg/ml, 0.6 μg/ml, 1.2 μg/ml, 5 μg/ml, 10 μg/ml, and 20 μg/ml in deionized water) were added into the dishes and incubated for 1 hour. The amount of adsorbed fibronectin was characterized by fluorescent staining using anti-fibronectin antibody (Sigma).

### Cardiomycyte Cultures

Primary ventricular cardiomyocytes were isolated from 1-day-old neonatal Wistar rats (Charles River Laboratories, MA) using a collagenase procedure as described previously (Aoki et al. 1998) and cultured on 0.1% gelatin-coated dishes. Cardiomyocyte culture medium consists of DMEM (Gibco, Gaithersburgh, Md.) with 10% FBS (Biowest, Miami, Fla.), 2 mM L-glutamine (Gibco), insulin/transferrin/selenious acid (ITS; 5 μg/mL, 5 μg/mL, 5 ng/mL, respectively) (Invitrogen), and 2% penicillin/streptomycin (Gibco). Murine 3T3-J2 fibroblasts (purchased from Howard Green, Harvard Medical School, Boston, Mass.), were maintained in 60-mm tissue culture dishes in DMEM plus 10% FBS and 2% penicillin and streptomycin. Rat heart microvascular endothelial cells (MVEC; purchased from VEC Technologies, Rensselaer, N.Y.), were maintained in DMEM supplemented with 10% FBS, 2 mM L-glutamine, ITS, 2% penicillin/streptomycin, and 10 ng/mL vascular endothelial growth factor (VEGF, R & D Systems, Minneapolis, Minn.). Culture medium was changed every three days.

### Fibroblast Cultures

Fibroblast were cultured in Dulbecco's Modified Eagle Medium (DMEM) High Glucose, 10% Fetal Bovine Serum (FBS) and 2% Penicillin/Streptomycin (P/S) at 37° C. and 5% CO2. The medium was changes every 2-3 days.

### Human Hepatocellular Liver Carcinoma Cell Line Cultures

The tissue culture dish was first coated with 0.1% gelatin at room temperature for 30-60 minutes. Once, the gelatin has been aspirated the human hepatocellular liver carcinoma cell line (HepG2) cells were seeded and cultured in DMEM High Glucose, 10% FBS, 2% P/S and 1% L-glutamine at 37° C. and 5% CO2. The medium was changed every 2-3 days.

### Liver Sinusoidal Endothelial Cell Cultures

Liver Sinusoidal Endothelial Cells (LSEC) medium consists of DMEM High Glucose, 10% FBS, 2% P/S, 40 μL Vascular Endothelial Growth Factor (VEGF), 400 μl L-glutamine,

1% Insulin-Transferrin-Selenium (ITS) solution. The cells were cultured at 37° C. and 5% CO2. The medium was changed every 2-3 days.

### Adult Primary Rat Hepatocyte Cultures

Adult primary rat hepatocytes (AH) medium consisted of DMEM High Glucose supplemented with 10% FBS, 2% P/S, 7 ng/ml glucagon, 7.5 μg/ml hydrocortisone, 0.5 U/ml insulin and 20 ng/ml EGF. The cells were cultured at 37° C. and 5% CO2. These cells cultured alone were used as the monocultures for comparison with co-culture models. The medium was changed daily and medium samples were collected for future functional analysis.

### Hepatocyte-Fibroblast Co-Cultures

Fibroblasts were maintained in P60 tissue culture dishes in fibroblast medium previously described in section 2.2.1. After reaching confluence, the fibroblasts were washed with PBS, trypsinized and plated into culture dishes in AH medium prior to seeding hepatocytes. AH cells were trypsinized and seeded into the previously prepared culture dishes with a cell seeding ratio of 1:1. The cell seeding density was typically 0.5×10 cells for a 12 well plate. Culture medium utilized for the co-cultures was AH medium and it was changed daily. Medium samples were collected for future functional analysis.

### Cell Seeding on 2-D Chitosan Films and 3-D Chitosan Nanofiber Scaffolds

For the 2-D cell cultures, the chitosan or chitosan/FN films were prepared in 24-well tissue culture dishes, as described in Section 2.2. Cells were seeded at a density of ˜25,000 cells/cm² on the films in 0.5 mL of culture medium. For 3-D cell cultures, the nanofibers were treated with fibronectin solution (10 μg/ml) for 1 hour to adsorb fibronectin on the fibers. The nanofibers were then soaked in culture medium for about 5 minutes prior to cell seeding. The suspended cells (fibroblasts, cardiomyocytes, or endothelial cells) in culture medium were directly seeded at a density of ˜300,000 cells/cm² on separate chitosan nanofibrous mats with 300 μl of medium and incubated for about 1 hour before adding further medium to allow for better cell entrapment and attachment. For the co-culture studies (cardiomyocytes-fibroblasts or cardiomyocytes-endothelial cells co-culture) in 2-D and 3-D culture systems, cells were seeded at the ratio of 1 to 1, giving a total initial cell number (cardiomyocytes+fibroblasts or cardiomyocytes+endothelial cells) of ˜50,000 cells/cm² for 2-D and ˜600,000 cells/cm² for 3-D cultures. The mats were incubated in a 10% CO₂, 37° C. incubator and the medium was changed every 3 days.

### Immunofluorescence Analysis

The samples were washed with PBS and fixed with 4% paraformaldehyde for 20 minutes at room temperature. After washing with PBS, the samples were permeabilized in 0.2% Triton X-100 in PBS for 10 minutes. The samples were then washed with PBS and incubated in blocking buffer (PBS/10% FBS/1% BSA) for 30 minutes. The primary antibody was then added and incubated for 60 minutes at room temperature. The samples were washed with PBS and then incubated with the secondary antibody for 60 minutes. The samples were then washed and examined with fluorescence microscopy (Nikon). The primary antibodies used were mouse anti-vinculin (Millipore, 1:200 dilution), mouse anti-α-sarcomeric actin (Invitrogen, 1:50 dilution), and rabbit anti-connexin 43 (Sigma, 1:1000 dilution). The secondary antibodies used were donkey anti-mouse IgG, alexa fluor 488 and donkey anti-rabbit IgG, alexa fluor 594 (Invitrogen). For the distribution of actin microfilaments, cells were stained by incubating fixed and permeabilized cultures with 0.1 μg/mL rhodamine phalloidin (Sigma) for 30 min. In some cases, cells were counterstained with 4, 6-diamidino-2-phenylindole (DAPI; Invitrogen) for nuclear staining. In order to verify the adsorption of fibronectin onto chitosan, the samples were stained for 1 hr at room temperature with rabbit anti-fibronectin. After washing twice with PBS, the samples were incubated with alexa fluor 488 conjugated anti-rabbit IgG, and washed twice with PBS. Cell viability on the nanofiber scaffolds was examined by a live/dead viability/cytotoxicity kit (Invitrogen).

### Scanning Electron Microscopy (SEM)

The cell seeded nanofibers scaffolds were examined with SEM. The samples were fixed with 2.5% glutaraldehyde for 24 hours at 4° C. The samples were then washed with PBS and serially dehydrated with 50%, 70%, and 100% ethanol for 15 minutes each. This was done to allow gradual dehydration of the cells preventing loss of cellular structural integrity. The samples were then vacuum-dried for about 6 hours. The samples were coated with carbon and observed with SEM.

### Quantitative Image Analysis

Nikon Imaging Solutions (NIS)-elements imaging software program (v. 3-448) was used to measure the diameter distribution of the chitosan fibers. The measurements were done from five different SEM images on different regions of each sample. A total of ˜150 measurements were analyzed for the fiber diameter. To quantify the amount of adsorbed fibronectin on chitosan surfaces, average fluorescence intensity were measured and analyzed after staining with anti-fibronectin, FITC. The experiments were performed at least twice in duplicate.

### Rheological Measurements of Chitosan Electrospinning Solution

The steady viscosity of the chitosan electrospinning solution was measured using Rheometric Scientific (T.A. Instruments) Stress Rheometer SR 200 25 mm PPS parallel plate at room temperature (25 C-30° C.). The solution was prepared as mentioned in the previous section and the viscosity measurements were performed at various time points after initial mixing of the chitosan in the TFA (2, 15, 24 hours and 7 days).

### Mechanical Tensile Testing

The stress-strain modulus and ultimate tensile strength were determined using an Instron uniaxial tensile testing equipment model 3343. The chitosan nanofiber matrices were prepared into rectangular strips (60×20 mm). The relative humidity was 20-30%, gauge was set to 20 mm and cross head speed of 10 mm/min was used. All the strips were tested until complete rupture with a 100N load cell at room temperature (25-30° C.).

### Fiber Swelling Assay

The chitosan nanofibers were prepared into squares (20×20×0.15 mm3) and neutralized in a basic solution composed of 15N ammonium hydroxide and 100% ethanol (1:1 v/v) for 30 minutes. The nanofibers were then washed with deionized water three times, each time for 10 minutes. The nanofibers were placed in phosphate buffer solution, PBS; pH 7.2 and incubated at 37° C. and 10% CO₂. The PBS solution was changed every 3 days. At certain time points (days 1, 7, 14, 21, 28) the nanofibers' diameter distribution was analyzed by SEM. The fiber diameters were quantified using Nikon Imaging Solutions (NIS)-elements basic research software (v. 3-448). Three chitosan sample duplicates was used for each time point and a total of ˜190 measurements were used to analyze the diameters.

### In Vitro Lysozyme Degradation Assay

The chitosan nanofiber matrices were prepared into squares (20×20×0.15 mm3) and neutralized as mentioned above. The degradation of the chitosan nanofibers was assessed by incubating them in egg-white lysozyme (MP Biomedicals LLC, CAT#100834) dissolved at 4 mg/ml in PBS, pH 7.2, at 37° C. and 10% CO₂. At specific time intervals (days 7, 14, 21, 28) the matrices were removed from the lysozyme solution, washed with water, vacuum dried for 24 hours, and weighed. The degradation profile was illustrated using the weight loss percentage of the dried sample before and after degradation.

### FTIR

Fourier transform infrared (FTIR) analysis was done using the Perkin Elmer FTIR-ATR 100 series. The spectra were analyzed in the 400-2000 cm-1 range with a resolution of 4 cm⁻¹ and 30 scan repeats. Unprocessed chitosan powder, film cast from the electrospinning solution and electrospun nanofibers were analyzed. The chitosan films were cast by pouring 2 ml of the chitosan electrospinning solution in a uncoated p35 petri dish and allowed to air dry in the chemical hood for over 48 hours.

### Thermal Analysis Via DSC, TGA

Thermogravimetric analysis (TGA, TA instruments model Q50, New Castle, Del.) was performed in an inert atmosphere (dry nitrogen, flow rate 40 ml/min), 10° C./min heating rate and maximum temperature of 300° C. Differential scanning calorimetry (DSC, TA instruments Q100, New Castle, Del.) was performed in sequential double scans on each sample. The first scan was to remove the water in the sample which was followed immediately by the second scan.

### Molecular Organization Analysis Via XRD

Chitosan powder, cast film and electrospun nanofibers were analysed by X-ray diffraction (XRD) using an X'pert Pro Diffractometer (PW3050/60, Philips, Netherlands). Monochromatized Cu K (1.54056A) X-ray source was used to irradiate the samples with a step size (2-theta) of 0.05°, scan step time of 1.0 sec and 2-theta range of 0-60°. The operating voltage and current were 45 kV and 40 mA, respectively.

### Albumin Assay

The culture medium samples were collected on Days 4, 8, 12, 18 and 22. The samples were stored at −20° C. for further analysis. The wells of a 96-well plate were incubated with albumin (5 mg/ml) in PBS overnight at 4° C. The plate was washed four times with 100 μl of PBS-Tween. Standard solutions in culture medium were prepared for 100, 50, 25, 12.5, 6.25, 3.125, 1.0625 and 0 μg/ml. 50 μl of standard solution and the samples were added to each well followed by the addition of Peroxidase conjugated sheep IgG anti-rat albumin (1:5000) in PBS-Tween and incubated overnight at 4° C. The plate was repeatedly washed four times with PBS-Tween. Substrate buffer consisting of 0.2M sodium phosphate and 0.1 M citric acid was prepared and 10 mg of o-phenylenediamine dihydrochloride (OPD) was dissolved in 25 ml of the buffer solution at room temperature. 10 μl of 30% hydrogen peroxide was added to the solution. The columns containing the samples were filled with 100 μl/well of the prepared solution at regular time intervals (approx. 10 seconds). The columns were then treated with 50 μl/well of 8N of sulfuric acid was added 5 mins after the initial start time. The absorbance was measure with a microplate reader at a wavelength of 450 nm or 490 nm.

### Ethoxyresorufin-O-dethylase Assay

Cytochrome P-450 A1 (CYPA1) enzymatic assay was assessed by measuring the ethoxyresorufin-O-dethylase (EROD) activity. The cultures were induced to produce CYPA1 by 2 μM of 3-methylcholanthren for 48 hours before measuring the EROD activity on Day 14 and Day 29. The cells were then washed well with PBS followed by 1 hour incubation with 8 μM ethoxyresorufin phenol free culture medium at 37° C. The medium is collected after incubation and the fluorescence intensity is measured. Resorufin was detected in the samples at an excitation wavelength of 530 nm and emission wavelength of 590 nm against resorufin standards using the fluorometer.

Although the systems and methods of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

1. A nanofiber 3-dimensional (3-D) scaffold which comprises an electrospun polymer fiber.

2. The scaffold of claim 1 which is a 3-dimensional (3-D) scaffold wherein the electrospun polymer fiber coated with surface coating molecule.

3. The scaffold of claim 2 wherein the surface coating molecule is a biomolecule.

4. The scaffold of claim 3 wherein the bimolecule is fibronectin, laminin, poly-lysine, collagen, glycosaminglycans, collagen, matrigel or gelatin.

5. The scaffold of claim 4 wherein the surface coating molecule is fibronectin.

6. The scaffold of claim 2 wherein the glycosaminglycans are ionically or covalently coated with a cross-linking reagent.

7. The scaffold of claim 6 where the cross-linking reagent is glutaraldehyde, genipin or EDC/sulfoNHS.

8-25. (canceled)
